SEQUENCE LISTING (21 sequences)

SEQ ID NO: 1 - DNA, 25 bp, Artificial Sequence
  GLI sequence
acagauucaggcucacgcuucdtdt

SEQ ID NO: 2 - DNA, 25 bp, Artificial Sequence
  GLI siRNA sequence
acagauucaggcucacgcuucdtdt

SEQ ID NO: 3 - DNA, 25 bp, Artificial Sequence
  GLI1 down- regulation
gcccuucaaagcccaguacaudtdt

SEQ ID NO: 4 - DNA, 25 bp, Artificial Sequence
  GLI1 down- regulation
auguacugggcuuugaagggcdtdt

SEQ ID NO: 5 - DNA, 25 bp, Artificial Sequence
  GLI1 down- regulation
ggcugcaccaaacgcuauacadtdt

SEQ ID NO: 6 - DNA, 25 bp, Artificial Sequence
  GLI1 down- regulation
uguauagcguuuggugcagccdtdt

SEQ ID NO: 7 - DNA, 26 bp, Artificial Sequence
  GLI1 down- regulation
ucuguauagcguuuggugcagcdtdt

SEQ ID NO: 8 - DNA, 26 bp, Artificial Sequence
  GLI1 down- regulation
gcugcaccaaacgcuauacagadtdt

SEQ ID NO: 9 - DNA, 26 bp, Artificial Sequence
  GLI1 down- regulation
aagauuggcgguguuccuuugcdtdt

SEQ ID NO: 10 - DNA, 26 bp, Artificial Sequence
  GLI1 down- regulation
gcaaaggaacaccgccaaucuudtdt

SEQ ID NO: 11 - DNA, 20 bp, Artificial Sequence
  CRISPR/Cas9 Sequences
agggagacccctaccggggc

SEQ ID NO: 12 - DNA, 25 bp, Artificial Sequence
  CRISPR/Cas9 Sequences
caccgagggagacccctaccggggc

SEQ ID NO: 13 - DNA, 25 bp, Artificial Sequence
  CRISPR/Cas9 Sequences
ctccctctggggatggccccgcaaa

SEQ ID NO: 14 - DNA, 20 bp, Artificial Sequence
  CRISPR/Cas9 Sequences
tcactcagcgagtgatgctt

SEQ ID NO: 15 - DNA, 25 bp, Artificial Sequence
  CRISPR/Cas9 Sequences
caccgaagcatcactcgctgagtga

SEQ ID NO: 16 - DNA, 25 bp, Artificial Sequence
  CRISPR/Cas9 Sequences
cttcgtagtgagcgactcactcaaa

SEQ ID NO: 17 - DNA, 210 bp, Artificial Sequence
  CRISPR/Cas9 Sequences
actcctgagtcccaagggctgtgggcaaggagctcaggaggagccggggagaccttgtcttgaccctctgacctcaggaccaccggggcagcgggagccagccgcagggagacccctaccggggctgggcgggaccactggccactgccagcctgtgtatccccgttggcaccccgcccaaacgggagctggggatcgaggcccctcctc

SEQ ID NO: 18 - DNA, 222 bp, Artificial Sequence
  CRISPR/Cas9 Sequences
ctcccacccaggcaaagctcccacccagttcccaagaagatccccagagtacacagactacaagactgcctctgcctctctgggacatcatttccccttacccctcccctcactcagcgagtgatgctttttttgttttgagacggagtctagctctgtcacccaggctggagtgcagtggcaccatctcggctcactgaaacctccgcctcccaggttcaa

SEQ ID NO: 19 - DNA, 764 bp, Artificial Sequence
  CRISPR/Cas9 Sequences
gcccagacagaggtgagaagggggggcaggcgggggaccacctgggagcagtgggggagggggcctgaggggatgctcagcttcttagggactcatcccagacccgggacatagaggcaaaataggggtgggagagcctggggtgagacattagaaactccagatttttcacttgtgtctttctctgtatcttctttttcttccctttttttcttctgtcagtctgtgtatctctgtctcagggaaccgtgggtctttgtctccgcctctcccatatattagaaatatcttactttcatgcggttaagtttaagaggctggagggatggctagctggaggtctgcgttgtagagaggtaaccccaggtgtgtgtctgcgcgtggggtaggaagatgtcagtgtttctgaaaggtggggactgcaaaggagggagctccaggtggggtggggacgggtgtgtgggaggcaacagagccactaggggccagccaggcttgaacctttgacctgtcttgtgacagatgtgccagtggatgcttgtgctttaggggaaaggagtgtcttctggacttggaagggggctggggcgggggggggctgtccaaggtctagtgaaggccctagaatgaccccatgcaatttggactcctgagtcccaagggctgtgggcaaggagctcaggaggagccggggagaccttgtcttgaccctctgacctcaggaccaccggggcagcgggagccagccgcagggagacccctac

SEQ ID NO: 20 - DNA, 801 bp, Artificial Sequence
  CRISPR/Cas9 Sequences
gagacggagtctagctctgtcacccaggctggagtgcagtggcaccatctcggctcactgaaacctccgcctcccaggttcaagcgattcttctgcctcagccttccgagtagctgggattacaggcacccgccatcatgactggctaatttttgtttttttgtagagacgggggtttcaccatgttggccaggctggtcttcaactgacctcaggtgatcctcccgcctcagcctctcaaagcgttggaattacaggcgtgagccactgtgcccggctcagtgatgctcttttcaactcgaattccgtggcagatgtcttagaggggtgggggataccagggatgttctgcccaggattctgtgcctgagactgctgtctgacagtctctatttcctccacctttatacctaccttccctttctgcagtgtccccacaccctcctctgagacgccatgttcaactcgatgaccccaccaccaatcagtagctatggcgagccctgctgtctccggcccctccccagtcagggggcccccagtgtggggacagaaggtcagtgtatataccaatccctgggccttgaggatttggcagatctcccacttgggcccacccccaccccatgccagtttcctatctacaggaggatttgaggccccatgtcatatggacctggaatctgggatcaggtcatgtctggggttttaaggtgaggtttatgtatcctccattcccattccagctgtctcttttttctaggactgtctggcccgcccttctgccaccaagctaacctc

SEQ ID NO: 21 - DNA, 423 bp, Artificial Sequence
  Blasticidin gene
atgaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaa

(19) United States
(12) Patent Application Publication
(10) Pub. No.: US 2022/0372486 A1
(43) Pub. Date: Nov. 24, 2022

(54) METHODS OF CANCER TREATMENT BY INHIBITION OF VASCULOGENESIS AND GLI1

(71) Applicant: Yekaterina Galat, Chicago, IL (US)

(72) Inventor: Yekaterina Galat, Chicago, IL (US)

(21) Appl. No.: 17/742,126

(22) Filed: May 11, 2022

Related U.S. Application Data
(60) Provisional application No. 63/187,176, filed on May 11, 2021.

(51) Int. Cl. C12N 15/113; A61K 38/46; A61K 31/7088; A61P 35/00

(52) U.S. Cl. CPC C12N 15/1135; A61K 38/465; A61K 31/7088; A61P 35/00; C12N 2310/14

(57) ABSTRACT

The present invention relates to methods and systems for a delay in the onset of early osteogenic markers, a reduction in the hematopoietic potential to form granulocyte units, and a decrease in vascular potential and in cancer-related gene expression. The present invention is a method of down-regulation of GLI1 via CRISPR or siRNA. The present invention indicates that the GLI1 intronic region is critical for the feedback loop and that GLI1 has lineage-specific effects on hESC differentiation. The present invention documents the extent of GLI1 abrogation on early stages of human development and to show that GLI1 transcription can be altered in a therapeutically useful way.

### CROSS-REFERENCE TO RELATED APPLICATIONS

The present application claims priority to U.S. Provisional Application Ser. No. 63/187,176 filed May 11, 2021, herein incorporated by reference in its entirety.

### SEQUENCE LISTING

The present application contains a Sequence Listing which is submitted electronically in ASCII format and is incorporated by reference in its entirety. Said ASCII copy, created on May 11, 2022, is named YKG101US_Sequencelisting_ST25 and 8 KB in size.

### BACKGROUND

This invention relates to methods and systems for genetic engineering and methods of cancer treatment.

The Sonic Hedgehog (SHH) signal transduction pathway is mediated by the GLI1, GLI2 and GLI3 transcription factors and plays roles in normal development¹⁻⁴. Following inhibition of the Patched (PTCH) receptor by the SHH ligand, the transmembrane protein Smoothened (SMO) releases its inhibition of GLI family transcription factors. GLI1 functions downstream of GLI2 and GLI3 during development, regulating cell proliferation and morphogenesis in many organ systems. In humans GLI1 inactivation is associated with a phenotypic spectrum extending from isolated postaxial polydactyly to an Ellis-van Creveld syndrome (EvC)-like condition⁵.

A significant cancer burden is associated with dysregulation of the SHH signal transduction pathway⁶⁻⁸. Indeed, Hedgehog signaling is believed to be active in up to one-third of all human cancers⁹⁻¹³. GLI1 gene targets sustain proliferation¹⁴, inhibit apoptosis¹⁴, promote angiogenesis^{15, 16} and promote tumor cell migration¹⁷. Furthermore, GLI1 expression is associated with chemotherapeutic drug resistance^{18,19}.

GLI1 is transcriptionally activated by GLI2.²⁰⁻²³. GLI1 is in a positive feedback loop with GLI2¹³ and likely with itself as it activates GLI1 reporters²⁴ with GLI1 and GLI2 driving further GLI1 expression. This feedback loop is considered to be an important element of the association of GLI1 expression with cancer phenotypes as described above since unrestrained it will continue to drive GLI1 expression, which is an oncogene. Negative regulation of the transcriptional feedback comes from GLI3, translational repression of GLI1, or lncRNA (GLI1as). The sequencing and public reference sequence data show six GLI binding sites (GBS) in the first intron of the human GLI1 gene. GLI1 and GLI2 bind the six GBS in this region and activate reporter expression. Elimination of some of the sites attenuates transcriptional activation of the transfected reporter construct. Removing the region containing all sites eliminates reporter gene activation. In addition, this region has an open chromatin configuration, and activating histone marks. In aggregate, these findings indicate that the six GBS are active cis-elements within a complex enhancer region and could regulate GLI1 expression²⁴ in vivo.

GLI1 is highly expressed in mesenchymal stem cells (MSCs)²⁵, neural stem cells (NSCs)²⁶, and embryonic stem cells (ESCs)²⁷. In MSCs, up-regulation of GLI proteins promotes osteogenic differentiation by inhibiting PPARγ and C/EBPα²⁵. Over-expression of SHH and/or GLI1 in human embryonic stem cells enhances production of neural progenitor and dopaminergic neurons. GLI1 up-regulates expression of Nanog, via binding Nanog regulatory sequences, which regulates self-renewal of NSCs²⁸. Interestingly, although loss of p53 activates the SIHH pathway and SHH downregulates p53, Po, et al. (2010) show that p53 is not required for SHH control of Nanog. The effects of p53 are mediated in part by competition with GLI1 for the transcriptional coactivator TAF9²⁹. Nanog in turn binds GLI proteins in ESCs and represses GLI1-mediated transcriptional activation. The expression profiles of GLI1 during differentiation and its function in stem cells are not yet clear.

Given the important role of GLI1 in cancer and development, the present invention attempts to address issues as well as others.

### SUMMARY OF THE INVENTION

Provided herein are systems and methods for gene editing the GLI1 gene.

The methods and systems are set forth in part in the description which follows, and in part will be obvious from the description, or can be learned by practice of the methods and systems. The advantages of the methods and systems will be realized and attained by means of the elements and combinations particularly pointed out in the appended claims. It is to be understood that both the foregoing general description and the following detailed description are exemplary and explanatory only and are not restrictive of the methods and systems, as claimed.

### BRIEF DESCRIPTION OF THE DRAWINGS

In the accompanying figures, like elements are identified by like reference numerals among the several preferred embodiments of the present invention.

FIGS. 1A-1L show the Generation and Characterization of GLI1 Edited H1 hESC. FIG. 1A is a schematic depiction of the strategy to target the region including the six GBS. Yellow triangles represent LoxP sites. FIG. 1B are microscope images (i-ix) of both the heterozygous (#5) and homozygous (#58) clones express green fluorescent protein (GFP) and have similar morphology as wild type hESCs. FIG. 1C are gel images of genotyping of heterozygous (#5 and #6) and homozygous (#56, #58, #64, and #65) H1 hESC clones by PCR-RFLP. Black arrows, PCR Primers; S, Scal; B, BamHI; Puro, Puromyocin, BSD, Blasticidin. FIG. 1D are western blot analysis showed decreased expression of GLI1 in heterozygous clone #6 (Het) and a dramatic reduction of GLI1 expression in homozygous clone #65 (Homo). Rh30 cells (human rhabdomyosarcoma cells) were used as a positive control for GLI1 protein. FIG. 1E are graphs of real-time PCR analysis showing that the deletion of this region of the GLI1 gene in the hESCs dramatically reduced the expression of GLI1 and its target, PTCH1. FIG. 1F are graphs showing the deletion of this region has minimal effects on the expression of pluripotency markers, OCT4, SOX2, and NANOG. FIG. 1G is a graph of real time PCR analysis showing that the GLI1 edited homozygous cells maintain higher pluripotency marker expression determined in embryoid bodies after 10 days of spontaneous differentiation. FIG. 1H is a graph of real time PCR analysis showing that the deletion of this region GLI1 in hESCs maintains reduction in expression of GLI1, but not GLI2 determined under conditions same as in (1G). FIG. 1I is a graph of real time PCR analysis showing that the deletion this region of the GLI1 gene in hESCs maintains reduction in expression of GLI1 targets determined under conditions same as in (1G). FIG. 1J-1L are graphs of real time PCR analysis showing that the deletion of this region of the GLI1 gene in hESCs significantly reduces the expression of markers of ectodermal (1J) mesodermal (1K), and endodermal (1L) lineages [*p<0.05, **p<0.01]. The rtPCR charts include data presented as mean±SEM from at least three independent experiments.

FIGS. 2A-2I show the assessment of the Effect of GLI1 Editing on Endothelial (Mesodermal) Differentiation Potential of hESCs. FIG. 2A are representative phase contrast images showing mesangioblast and mesenchymal colonies growing in semisolid media generated from WT H1 hESC (blue circle) and GLI1 edited clones (#6 and #65) (red circles). [Right panel] Higher magnification of mesengioblast (BL) and mesenchymal (MS) colonies. FIG. 2B is a graph showing a significant increase in the number of MS colonies in GLI1 edited clone #65 (homozygous), but no significant increase in the number of MS colonies in GLI1 edited clone #6 (heterozygous) compared to the WT H1 hESC during the early mesodermal specification; Bars represent mean±SEM from three independent experiments [**p<0.001]. FIGS. 2C-2D are graphs and representative flow cytometry analysis showing that the endothelial differentiation efficiency of homozygous GLI1 edited clones is significantly lower than the WT H1 hESC (H1). Bars represent mean±SEM from three independent experiments. FIGS. 2E-2F are graphs and representative flow cytometry analysis and a graph demonstrating high cell survival during endothelial differentiation. There was no significant difference in survival between the WT H1 hESC and GLI1 edited clones. FIG. 2G is a volcano plot showing the RNA-Seq data distribution for endothelial differentiation of WT H1 hESC vs. GLI1 edited cells. In relation to WT H1 hESC, the down-regulated genes are on the left and up-regulated genes are on the right. FIG. 2H is a heatmap showing vascular-related gene expression comparison between WT H1 hESC (H1) and GLI1 edited clone (#65). The expression, p-value, and fold change data for all gene tables was obtained from RNA-Seq analysis. FIG. 2I is a heatmap showing cancer-related gene expression comparison between WT H1 hESC (H1) and GLI1 edited cells (#65).

FIGS. 3A-3D show the assessment of the Effect of GLI1 Editing on Hematopoietic (Mesodermal) Differentiation Potential of hESCs. FIG. 3A is a representative phase contrast images showing myeloid colonies growing in semisolid media. FIG. 3B is a graph demonstrating the number of myeloid colonies generated from WT H1 hESC (H1) and GLI1 edited cells (GLI1 6 and GLI1 65). Bars represent mean±SEM from three independent experiments; G: granulocyte colony forming unit, GM: granulocyte macrophage colony forming unit [*p<0.05]. FIG. 3C is a representative phase contrast images showing granulocyte colonies growing in semisolid media after the siRNA treatment. FIG. 3D is a graph demonstrating the number of myeloid colonies generated from WT H1 hESC (H1) treated with scrambled siRNA and WT H1 hESC (H1) treated with siRNA combination (1+2). Bars represent mean±SEM from three independent experiments; G: granulocyte colony forming unit, GM: granulocyte macrophage colony forming unit [*p<0.05].

FIGS. 4A1-4F show the assessment of the Effect of GLI1 Editing on Osteogenic (Mesodermal) Differentiation Potential of hESCs. FIGS. 4A1-4A3 are graphs showing the time course of gene expression in WT H1 hESC (H1) and the GLI1 homozygous edited cells (GLI1 65) during osteogenic differentiation as determined by real-time PCR. Average expression was normalized to GAPDH, shown as mean±SEM from at least three independent experiments [(ALPL, **p<0.0001), (RNX2, *p=0.0019, **p=0.004), BGLAP, *p=0.0138, **p=0.0015)]. FIGS. 4B-4C are graphs and representative images showing that GLI1 homozygous edited cells had a significant delay in mineralization at day 8 (D8) of osteogenic differentiation [**p=0.000007]. By day 10 (D10), there was no significant difference. FIG. 4D is a graph of the MTS assay showing a significant difference in cell viability between WT H1 ESC and GLI1 edited cells during osteogenic differentiation [**p<0.0001]. FIG. 4E is a volcano plot showing the statistical significance of RNA-Seq data of osteogenic differentiation. FIG. 4F is a heatmap showing osteogenic gene expression comparison between WT H1 hESC and GLI1 edited cells. The expression, p-value, and fold change data for all gene tables was obtained from RNA-Seq analysis.

FIGS. 5A-5F show the assessment of the Effect of GLI1 Editing on Endodermal and Ectodermal Differentiation Potential of hESCs. FIG. 5A is a graph of the real-time PCR analysis of the GLI1 homozygous edited cells (GLI1 65) showed dramatically reduced expression GATA-6 during endodermal differentiation as compared to WT H1 hESC on day 3 (D3) of differentiation [**p<0.0001]. FIG. 5B is a volcano plot showing the statistical significance of RNA-Seq data at day 5 of endodermal differentiation. The expression, p-value, and fold change data for all gene tables was obtained from RNA-Seq analysis. FIG. 5C is a heatmap showing endodermal gene expression comparison between WT H1 hESC and GLI1 edited cells. FIG. 5D is a representative images of neurospheres formed by the WT H1 NPCs (i, iii) and representative images of neurospheres formed by the NPCs from GLI1 edited cells (ii, iv). Representative image showing neurosphere outlines for quantitation prior to area calculations performed via the ImageJ program (v). Graphical depiction of average area (reported in pixels) covered by H1 and GLI1 edited neurospheres (vi). The results were from 8 experimental trials and reported as ±SEM [*p=0.030]. FIG. 5E is a real-time PCR analysis showing that editing the first intron of the GLI1 gene in the hESCs dramatically reduced the expression of neural markers during day 7 of neural differentiation [**p<0.0001]. FIG. 5F is a volcano plot showing the statistical significance of RNA-Seq data of neural differentiation. FIG. 5G is a heatmap showing neural gene expression comparison between WT H1 hESC and GLI1 homozygous edited cells during day 28 of neural differentiation. FIG. 5H is a representative phase contrast and immunofluorescent images showing positive expression of early neural marker Nestin and late neural marker GFAP (green) and MAP2 (red) in GLI1 homozygous edited cells.

FIGS. 6A1-6B2 show the assessment of the Effect of GANT-61 Differentiation of hESCs. FIGS. 6A1-6A2 are graphs showing the expression of pluripotency markers, GLI1, PTCH1, and Smoothened (SMO) following 5 μM and 20 μM GANT-61 treatment of hESCs maintained in mTeSR1 media. There were no significant differences in expression of these markers after 6 and 12 days following GANT-61 addition to mTeSR1. FIGS. 6B1-6B2 are graphs in the early stage of spontaneous differentiation in embryoid bodies (Day 10), GANT-61 treatment down-regulated the expression of pluripotency genes, GLI1, GLI2, PTCH1, and Plakoglobin. Ectodermal differentiation was also inhibited. On the other hand, GANT-61 treatment promoted mesodermal and endodermal differentiation in H1 hESCs. At a later stage (Day 20), GANT-61 treatment continued to down-regulate the expression of pluripotency genes, GLI1, GLI2, and PTCH1 as well as significantly reduced markers of mesodermal and endodermal differentiation.

FIGS. 7A-7C show the activation of Reporter Expression by Human Transcription Factors GLI1, GLI2, GLI1-AT and tGLI1. FIG. 7A show the partial map of genomic organization (left) of GLI1 vs. GLI-AT and tGLI1. Partial map of plasmid construct (right). FIG. 7B show the luciferase expression of reporter construct with co-transfection of GLI1, GLI1-AT or tGLI1 with the full length 1st intron (details in Taylor, et al, 24). FIG. 7C show the reporter expression following co-transfection of GLI1 and GLI2 in combination. Relative concentrations shown on x-axis.

FIG. 8A show the schematic of GLI1 First Intron Deletion Constructs. FIG. 8B show the 6 GBS sites are shown above along with the primer positions for the deletions. Deletions are shown in brackets. The effect of the deletion on reporter expression is shown with the symbol “X” for co-transfection with either GLI1 or GLI2. Derived from Taylor, et al (24).

FIGS. 9A-9B are graphs showing the GLI1 Intron1a Controls Autoregulation of GLI1 Transcription. GLI1 (FIG. 9A) and GLI2B (FIG. 9A) activate expression of a luciferase reporter construct with the GLI1 intron1a upstream of a minimal promoter. This activity is lost when 6 GLI binding sites are deleted from the regulatory intron. Stars indicate significance: p-value <0.001; compared to empty reporter vector by student's t-test.

FIG. 10 are graphs showing GLI2 expression Does Not Compensate for GLI1 Down-Regulation. According to rtPCR data, GLI2 levels remained constant throughout directed differentiation experiments. Furthermore, the lack of significant differences in expression between GLI1 vs. H1 hESC differentiated control further confirms that GLI2 does not promote differentiation toward any particular lineage when GLI1 is down-regulated.

FIGS. 11A-11C2 show Hematoendothelial Differentiation (MetaCore Map IDs: 4857, 4860) and Assessment (Related to FIGS. 2 and 3). FIG. 11A is a schematic pathway interaction symbols (obtained from MetaCore Reference Guide). FIG. 11B is a graph compared to H1 hESCs, the pathway maps indicate that several receptors and genes involved in promoting lymphatic, venous, and arterial endothelial differentiation are down-regulated in GLI1-edited cells (GLI1 65). Hematopoietic differentiation potential also exhibits a down-regulatory effect in the GLI1 65 clone. Arrows refer to down-regulated expression levels, and the stars indicate significance (p-values <0.0001). FIGS. 11C1-11C2 is a schematic showing H1 hESCs were treated with a combination of 2 siRNAs. GLI1 down-regulation was observed during days 2-4 following treatment.

FIG. 12 is a schematic showing the Osteogenic Differentiation (MetaCore Map ID: 4363 (Related to FIG. 4)). The interconnection of WNT and Notch signaling pathways shows the decreased osteogenic differentiation potential of the GLI1-edited cells (GLI1 65) in comparison to H1 hESCs. The up-regulated expression levels of PPAR-gamma inhibit RUNX2 expression, which decreases the downstream expression of osteogenic genes Osteopontin, COL1A1, and ALPL. The arrows indicate up- and down-regulation. The stars designate expression level significance (p-values <0.0001).

FIG. 13 is a schematic showing the Endodermal Differentiation (MetaCore Map IDs: 4788, 4867, 4868) (Related to FIG. 5). The gene expression data shows that GLI1-edited cells (GLI1 65) have a decreased predisposition toward endodermal differentiation in comparison to H1 hESCs. SOX17, a downstream NODAL signaling target, is down-regulated in the GLI165 clone. The hindered ability of these cells to progress toward mesendoderm formation should prevent subsequent endodermal lineage commitment. This correlates with the down-regulation of BMP signaling targets GATA-2 and GATA-6, which affect endodermal differentiation. The arrows indicate down-regulation, and stars designate expression level significance (p-values <0.0001).

FIGS. 14A-14B is a schematic showing the Neural Differentiation (MetaCore Map ID: 4788) (Related to FIG. 5). In comparison to H1 hESCs, the early differentiation stage of GLI1 65 shows more neural progenitor markers are up-regulated vs late stage differentiation. The arrows indicate up- and down-regulation. Stars designate expression level significance (p-value <0.002).

FIG. 15 is a graph showing the GANT-61 Inhibition of GLI Target Genes (Related to FIG. 6). Rh18 and Rh41 cell lines exhibit increased GLI1 expression. Following treatment with GANT-61, these cell lines exhibited significant down-regulation of BCL-2 promoter expression, which is activated in response to GLI1 signaling.

FIGS. 16A-16B are schematics showing the GLI1 Transcriptional Targets (Related to FIG. 1). FIG. 16A. Complex containing the GLI1 oncogene (chain A) bound to the high-affinity DNA binding site via five Zn fingers. The structure was obtained from the PDB database (accession #: 2GLI). Out of the three GLI1 transcription factors, only GLI1 directly activates the expression of HHIP, SPP1, CCND2, MYCN, and IGFBP6. The rtPCR data shows that these genes are down-regulated in the GLI1 knockdown clone (GLI1 65). Arrows refer to down-regulated expression levels, and the stars indicate significance (p-values <0.0001). FIG. 16B GLI1 targets pathway map created via MetaCore's Pathway Map Creator application. The HHIP gene is up-stream of GLI1, and the additional target genes (MYCN, CCND2, SPP1, IGFBP6) are down-stream.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the invention the terms “chimeric RNA”, “chimeric guide RNA”, “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”. An exemplary CRISPR-Cas system is indicated below.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “species” are used interchangeably herein to refer to a vertebrate, preferably a mammal. Mammals include, but are not limited to, murines, simians, humans, farm animals, sport animals, and pets.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possesses catalytic activity for DNA cleavage. A cleavage domain can be contained in a single polypeptide chain or cleavage activity can result from the association of two (or more) polypeptides.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). IRES may be substituted for P2A. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EFI1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Promoters/enhancers which may be used to control the expression of a shRNA construct in vivo include, but are not limited to, the PolIII human or murine U6 and H1 systems, the cytomegalovirus (CMV) promoter/enhancer, the human 3-actin promoter, the glucocorticoid-inducible promoter present in the rat and mouse mammary tumor virus long terminal repeat (MMTV LTR), the long terminal repeat sequences of Moloney murine leukemia virus (MuLV LTR), the SV40 early or late region promoter, the promoter contained in the 3′ long terminal repeat of Rous sarcoma virus (RSV), the herpes simplex virus (HSV) thymidine kinase promoter/enhancer, and the herpes simplex virus LAT promoter. Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., an immunoglobulin promoter, and from heat-shock promoters, provided such promoters are compatible with the host cell systems. Inducible systems, such as Tet promoters may be employed. In addition, recombinase systems, such as Cre/lox may be used to allow excision of shRNA constructs at desired times. The Cre may be responsive (transcriptionally or post-transcriptionally) to an external signal, such as tamoxifen.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

The terms “reduce”, “reduction”, or “decrease” of expression of a gene or gene product (e.g., RNA or protein) refer to an overall decrease of at least 25%, 30%, 40%, 50%, 60%, 70%, 80%, 81%, 82%, 83%, 84%, 85%, 86%, 87%, 88%, 89%, 90%, 91%, 92%, 93%. 94%, 95%, 96% 97%, 98% or 99% up to 100% (abrogation or elimination) in the transcription and/or translation of a gene or in the levels of the gene product (e.g., RNA or protein).

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

“Recombinase Mediated Cassette Exchange” (RMCE) is based on the features of site-specific recombination processes (SSRs), the procedure permits the systematic, repeated modification of higher eukaryotic genomes by targeted integration. For RMCE, this is achieved by the clean exchange of a preexisting gene cassette for an analogous cassette carrying the “gene of interest” (GOI). The exchange of genetic cassettes (‘flip’ step) is enabled by a recombinase (‘Flp’) from yeast. Part B shows mutants (Fn) of the naturally occurring 48 bp FRT-site (F). If a gene cassette is flanked by a set of these sites (F and Fn, for example) it can change places, by double-reciprocal recombination, with a second cassette that is part of an exchange plasmid. A model experiment is shown in part C, in which an ‘empty’ cell is modified by either a standard transfection approach or by RMCE. Please note that in the first case multiple genomic sites are hit, each giving raise to a different expression level (cf. the broad distribution of green dots). If a pre-defined genomic address is used to introduce the same gene reporter, each clone derived from such an event shows comparable expression characteristics.

“Recombinases” are genetic recombination enzymes. DNA recombinases are widely used in multicellular organisms to manipulate the structure of genomes, and to control gene expression. These enzymes, derived from bacteria and fungi, catalyze directionally sensitive DNA exchange reactions between short (30-40 nucleotides) target site sequences that are specific to each recombinase. These reactions enable four basic functional modules, excision/insertion, inversion, translocation and cassette exchange, which have been used individually or combined in a wide range of configurations to control gene expression.

The “tet inducible system” is a method of inducible gene expression where transcription is reversibly turned on or off in the presence of the antibiotic tetracycline or one of its derivatives (e.g. doxycycline). In nature, the Ptet promoter expresses TetR, the repressor, and TetA, the protein that pumps tetracycline antibiotic out of the cell. The difference between Tet-On and Tet-Off is not whether the transactivator turns a gene on or off, as the name might suggest; rather, both proteins activate expression. The difference relates to their respective response to doxycycline (Dox, a more stable tetracycline analogue); Tet-Off activates expression in the absence of Dox, whereas Tet-On activates in the presence of Dox. The Tet-On Advanced transactivator (also known as rtTA2S-M2) is an alternative version of Tet-On that shows reduced basal expression, and functions at a 10-fold lower Dox concentration than Tet-Off. In addition, its expression is considered to be more stable in eukaryotic cells due to being human codon optimized and utilizing 3 minimal transcriptional activation domains. Tet-On 3G (also known as rtTA-V16[Clontech Laboratories, Inc.]) is similar to Tet-On Advanced but was derived from rtTA2S-S2 rather than rtTA2S-M2. It is also human codon optimized and composed of 3 minimal VP16 activation domains. However, the Tet-On 3G protein has 5 amino acid differences compared to Tet-On Advanced which appear to increase its sensitivity to Dox even further. Tet-On 3G is sensitive to 100-fold less Dox and is 7-fold more active than the original Tet-On. Other systems such as the T-REx system by Life Technologies work in a different fashion. The gene of interest is flanked by an upstream CMV promoter and two TetO2 sites. Expression of the gene of interest is repressed by the high affinity binding of TetR homodimers to each TetO2 sequences in the absence of tetracycline. Introduction of tetracycline results in binding of one tetracycline on each TetR homodimer followed by release of TetO2 by the TetR homodimers. Unbinding of TetR homodimers and TetO2 result in derepression of the gene of interest.

“Transduction of foreign DNA material” is the process by which genetic material, e.g. DNA or siRNA, is inserted into a cell by a virus. Common techniques in molecular biology are the use of viral vectors (including bacteriophages), electroporation, or chemical reagents that increase cell permeability. Transfection and transformation are also common ways to insert DNA into a cell.

“Blastocyst injection” generate of chimeric rat, i.e. mixtures of ES cell-derived and host blastocyst-derived tissues. The goal is a chimera with high contribution of ES cell-derived tissue, including the germline. ES cells for injection can be prepared. Blastocysts (from strain C57BL/6 for 129-derived ES cells; from strain albino C57BL/6 for C57BL/6-derived ES cells) may be injected with gene-modified ES cells and implanted into recipient dams. Chimeric males may then be used for experimentation.

A variety of cells isolated or obtained from other sources (e.g., commercial sources or cell banks), can be used in accordance with the invention. Non-limiting examples of such cells include somatic cells such as immune cells (T-cells, B-cells, Natural Killer (NK) cells), blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, thymic nurse cells, Schwann cells, etc.). Eukaryotic germ cells (spermatocytes and oocytes) can also be used in accordance with the invention, as can the progenitors, precursors and stem cells that give rise to the above-described somatic and germ cells. These cells, tissues and organs can be normal, or they can be pathological such as those involved in diseases or physical disorders, including but not limited to immune related diseases, chronic inflammation, autoimmune responses, infectious diseases (caused by bacteria, fungi or yeast, viruses (including HIV) or parasites), in genetic or biochemical pathologies (e.g., cystic fibrosis, hemophilia, Alzheimer's disease, schizophrenia, muscular dystrophy, multiple sclerosis, etc.), or in carcinogenesis and other cancer-related processes. Rat pluripotent cells, including embryonic cells, spermatogonial stem cells, embryonic stein cells, and iPS cells are envisioned. Rat somatic cells are also envisioned.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The terms “CRISPR system,” “Cas system” or “CRISPR/Cas system” refer to a set of molecules comprising an RNA-guided nuclease or other effector molecule and a guide RNA (gRNA) molecule that together are necessary and sufficient to direct and effect modification of nucleic acid at a target sequence by the RNA-guided nuclease or other effector molecule. In some embodiments, a CRISPR system comprises a gRNA and a Cas protein, e.g., a Cas9 protein. In some embodiments, a CRISPR system comprises two or more gRNAs and a Cas protein, e.g., a Cas9 protein. Such systems comprising a Cas9 or modified Cas9 molecule are referred to herein as “Cas9 systems” or “CRISPR/Cas9 systems.” In one example, the gRNA molecule and Cas molecule may be complexed, to form a ribonuclear protein (RNP) complex.

The terms “Cas9” “Cas9 protein” or “Cas9 molecule” refer to an enzyme from bacterial Type II CRISPR/Cas system responsible for DNA cleavage. Cas9 used herein also includes wild-type protein as well as functional and non-functional variants thereof.

The term “gene editing nuclease” as used herein refers to a polypeptide or protein comprising one or more DNA-binding domains or components and one or more DNA-cutting domains or components. The term also encompasses isolated nucleic acids, e.g., one or more vectors, encoding said DNA-binding and DNA-nuclease domains or components. Gene editing nucleases are used for modifying the nucleic acid of a target gene and/or for modulating the expression of a target gene. For example, the one or more DNA-binding domains or components are associated with the one or more DNA-cutting domains or components, such that the one or more DNA-binding domains target the one or more DNA-cutting domains or components to a specific nucleic acid site. Gene editing nuclease that can be used in the present disclosure include but are not limited to, Clustered Regularly Interspaced Short Palindromic Repeats (CRISPR)/Cas nucleases, zinc finger nucleases (ZFN), transcription activator-like effector nucleases (TALENs), and meganucleases.

The terms “guide RNA”, “guide RNA molecule”, “gRNA molecule” or “gRNA” are used interchangeably, and refer to a set of nucleic acid molecules that promote sequence-specific targeting of an RNA-guided nuclease or other effector molecule (typically in complex with the gRNA molecule) to a target sequence. In some embodiments, targeting is accomplished through hybridization of a portion of the gRNA to DNA (e.g., through the gRNA targeting domain), and by binding of a portion of the gRNA molecule to the RNA-guided nuclease or other effector molecule (e.g., through at least the tracrRNA). In some embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA”, and the like. gRNA molecules are described in more detail below, but generally include a targeting domain and a tracrRNA. In some embodiments the targeting domain and tracrRNA are disposed on a single polynucleotide. In other embodiments, the targeting domain and tracr are disposed on separate polynucleotides.

RNA interference (RNAi)-related technique is the use of active components of RNAi are short/small double stranded RNAs (dsRNAs) called small interfering RNAs (siRNAs). As a non-limiting example, the RNAi-related technique may comprise siRNA molecules, e.g. siRNA duplexes, targeting a GLI1, which may be designed and synthesized in vitro and introduced into cells to activate RNAi. According to the present disclosure, one or more siRNA molecules that target GLI1 are designed and disclosed herein. In some embodiments, one or more siRNA molecules

### DESCRIPTION OF EMBODIMENTS

In some embodiments, the GLI1 expression has been knocked out. As a non-limiting example, the GLI1 expression has been knocked out by Clustered Regularly Interspaced Short Palindromic Repeats (CRISPR)-Cas nuclease, e.g. a CRISPR/Cas9 nuclease, a zinc finger nuclease (ZFN), a transcription activator-like effector nuclease TALEN nuclease, or a meganuclease. In some embodiments, the GLI1 expression has been knocked down. As a non-limiting example, the GLI1 expression has been knocked down by an RNA interference (RNAi)-related technique. As a non-limiting example, the RNAi-related technique may be a short hairpin RNA (shRNA). A short hairpin RNA or small hairpin RNA (shRNA/HIairpin Vector) is an artificial RNA molecule with a tight hairpin turn that may be used to silence target gene expression via RNAi. The shRNAs may be incorporated into plasmid vectors and integrated into genomic DNA for long-term or stable expression, for extended knockdown of the target mRNA.

In some embodiments, the GLI1 inhibitory molecule that is modulated or is reduced in expression in the population of cells comprising GLI1 modified cells. In some embodiments, the GLI1 expression has been knocked out. In some embodiments, the GLI1 expression has been knocked out by a Clustered Regularly Interspaced Short Palindromic Repeats (CRISPR)-Cas nuclease, e.g. a CRISPR/Cas9 nuclease, a zinc finger nuclease (ZFN), a transcription activator-like effector nuclease TALEN nuclease, or a meganuclease. In some embodiments, the GLI1 expression has been knocked out by a CRISPR/Cas nuclease. In some embodiments, the GLI1 expression has been knocked out by a CRISPR/Cas9 nuclease. In some embodiments, the knockout of GLI1 expression in the cells generates a population of cells comprising GLI1-modified cells

In some embodiments, the GLI1 expression is knocked down. In some embodiments, the GLI1 expression may be knocked down by an RNA interference (RNAi)-related technique. The active components of RNAi are short/small double stranded RNAs (dsRNAs) called small interfering RNAs (siRNAs). As a non-limiting example, the RNAi-related technique may comprise siRNA molecules, e.g. siRNA duplexes, targeting a GLI1, which may be designed and synthesized in vitro and introduced into cells to activate RNAi. According to the present disclosure, one or more siRNA molecules that target GLI1 are designed. In some embodiments, one or more siRNA molecules used in the methods described herein comprise the nucleotide sequence of any one of SEQ ID NO: 1-3. In some embodiments, one or more siRNA molecules used in the methods described herein consist of the nucleotide sequence of any one of SEQ ID NO: 1-3. In some embodiments, the knockdown of GLI1 expression in the cells generates a population of cells comprising GLI1 modified cells to treat the disease or disorder.

GLI1 is one of three GLI family transcription factors that mediate Sonic Hedgehog signaling, which plays a role in development and cell differentiation. GLI1 forms a positive feedback loop with GLI2 and likely with itself. To determine the impact of GLI1 and its intronic regulatory locus on this transcriptional loop and human stem cell differentiation, the region containing six GLI binding sites in the human GLI1 intron were deleted using CRISPR/Cas9 editing to produce H1 human embryonic stem cell (hESC) GLI1-edited clones. Editing out this intronic region, without removing the entire GLI1 gene, allowed the effects of this highly complex region to be determined, which binds transcription factors in a variety of cells.

The roles of GLI1 in human ESC differentiation were investigated by comparing RNA-Seq, q-rtPCR, and functional assays. Editing this region resulted in GLI1 transcriptional knockdown, delayed neural commitment, and inhibition of endodermal and mesodermal differentiation during spontaneous and directed differentiation experiments. Given the important role of GLI1 in cancer and development, a stem cell model was established to study the relationship between the region containing the six GBS in the GLI1 first intron, GLI1 expression, and the functions of GLI1 during early stages of embryonic stem cell development. In the null GLI1 mouse model, GLI2 apparently can compensate, explaining why GLI1 KO animals do not seem to develop an obvious phenotype 30-32. The approach of editing out the GLI1 intronic region without removing the entire GLI1 gene allowed to investigate the effects of this highly complex region, which binds many trans factors in a wide variety of cell types. The regulatory region containing the six GBS regulates GLI1 expression in turn affects differentiation of ESCs.

A combination of spontaneous differentiation in embryoid bodies, directed differentiation, small molecule inhibitors and RNA-Seq were employed to gain insight into the developmental roles of this regulatory region. The six GBS in the first intron bind GLI1 and GLI2 transcription factors and that the binding activates transcription. In vivo CRISPR/Cas9 editing reduces GLI1 expression to barely detectable levels and significantly affects GLI1 target genes. This results in lineage specific effects on differentiation. Abrogation of GLI1 expression makes the region and the protein complexes that occupy it attractive therapeutic targets, particularly in cancer.

CRISPR Use in Therapy:

First-in-human in vivo application of gene editing tools based on CRISPR-Cas9 have been used for the treatment of inherited retinal dystrophies (Pulman et al., 2022).

The detailed description of CRISPR/Cas design, methods for deletion of the first intron of Gli1 in hESCs and clone selection in vitro is provided in the application. For in vivo delivery to the patient plasmid DNA, lentivirus, or adeno-associated virus (AAV) may be used as a vehicle for CRISPR. Lentivirus and plasmid DNA may be used for local administration. AAV can be injected locally or systemically into the circulatory system through intravenous injection where expression of the gene editing toolkit can be controlled to target specific organs via tissue-specific promoters.

AAV will be used since it is currently approved for >80 diseases and has been used in clinical trials. Because of the limited size capacity of the AAV vector for gene delivery, a dual AAV5 system will be used, with one carrying the Staphylococcus aureus Cas9 and the other carrying the guides sgRNA. The DSB induced by Cas9 and both guides result in deletion of the Gli1 intronic region. Both sgRNAs and Cas9 nuclease will be cloned into puromycin-resistant vectors. Mixtures of the 2 AAVs will be injected to the site of tumor location or its proximity.

For glioblastoma, local administration will be used by stereotaxic injection into the lateral ventricle or the hippocampus.

siRNA Use in Therapy Example:

siRNA is widely used in tumor gene therapy research and siRNA-based therapeutics are growing (Zhang et al., 2021). siRNA will be used to suppress the expression of Gli1 with the aim of 1) inhibition of tumor anti-apoptosis genes, and 2) inhibition of tumor angiogenesis-related factors.

The detailed description of siRNA sequence, and methods of siRNA in vitro use for Gli1 interference in hESCs is provided herein.

For in vivo use some of the known chemical modifications to the backbone, base, or sugar of the RNA can been considered to enhance siRNA stability. Similarly, some of the known delivery system, for instance, liposomes or exosome loaded siRNA can be used that enhances siRNA cell uptake. Exosomes can effectively cross the blood-brain barrier and can be used for the treatment of glioblastoma. The method of exosome production for treatment of cancer cells in vitro was earlier described by (Khalkhali-Ellis et al., 2016). siRNA will be injected to the site of tumor location or its proximity. For glioblastoma, local administration by stereotaxic injection into the lateral ventricle or the hippocampus will be used.
Khalkhali-Ellis, Z., Galat, V., Galat, Y., Gilgur, A., Seftor, E. A., Hendrix, M. J. C., 2016. Lefty Glycoproteins in Human Embryonic Stem Cells: Extracellular Delivery Route and Posttranslational Modification in Differentiation. Stem Cells Dev 25, 1681-1690.
Pulman, J., Sahel, J. A., Dalkara, D., 2022. New Editing Tools for Gene Therapy in Inherited Retinal Dystrophies. Crispr j.
Zhang, M. M., Bahal, R., Rasmussen, T. P., Manautou, J. E., Zhong, X. B., 2021. The growth of siRNA-based therapeutics: Updated clinical studies. Biochem Pharmacol 189, 114432.

### Example 1

Materials and Methods

Cell Line

H1 (WA01), a well characterized human embryonic stem cell line, 33 was purchased from WiCell (Madison, Wis., https://www.wicell.org/home/stem-cells/catalog-of-stem-cell-lines/wa01.cmsx). The cells were maintained on Matrigel-coated culture dishes in mTeSR1 medium (STEMCELL Technologies) or StemMACS iPS-Brew XF (Miltenyi Biotec).

Generation of CRISPR Plasmids

sgRNAs for the upstream and downstream sequences of the six GLI binding sites were designed using Optimized CRISPR Design (From Dr. Feng Zhang's lab in Massachusetts Institute of Technology, USA. http://crispr.mit.edu/) and Cas-Designer (From Dr. Jin-Soo Kim's Lab in Seoul National University, South Korea. http://www.rgenome.net/cas-designer/). The oligos were annealed and cloned into px330 vector (Addgene, Plasmid #42230). The upstream and downstream homolog arms were amplified by PCR with Q5® High-Fidelity DNA Polymerase (M0491S, New England Biolab (NEB), MA, USA) and cloned into HR700PA-1 Gene Knock-out Targeting Vector (System Biosciences (SBI), CA, USA) with NEBuilder® HiFi DNA Assembly Master Mix (E2621S, NEB). The sequences were verified by Sanger sequencing (GENEWIZ, NJ, USA). To generate Targeting Vector carrying Blasticidin, the Puromycin resistance gene was replaced with Blasticidin gBlocks Gene Fragment.

Generation of Edited H1 ESCs

A total amount of 2 ug DNA has been used for the transfection. Plasmids were co-transfected into H1 ESCs with the Human Stem Cell Nucleofector™ Kit 1 (VAPH-5012, Lonza) in a Nucleofector™ 2b device. H1 hESCs cell were cultured and the potential positive clones collected. After 72 hours, 10 μg/ml Puromycin dihydrochloride (NA0310, Sigma) was added into the culture media. Genomic DNA of transfected H1 hESCs was isolated with PureLink® Genomic DNA Kits. Genotyping of transfected H1 hESC clones was performed with DreamTaq Green PCR Master Mix according to the manufacturer's protocol.

To generate homozygous edited H1 hESCs, a targeting vector carrying Blasticidin was co-transfected with a px330 vector carrying sgRNA by using the Nucleofector™ 2b device. After 72 hours, 5 μg/ml Blasticidin S HCL (Thermo Fisher Scientific) and 10 μg/ml Puromycin dihydrochloride were used to select the double edited clones.

GLI1 Western Blot Analysis

The following procedures were carried out at 4° C. Approximately 1×10⁶ cells were re-suspended and incubated in 0.15 ml of RIPA buffer (Sigma) and protease inhibitor cocktail (Pierce) for 5 min with rocking. The protein concentration was determined with BCA assay kit (Pierce). All the following procedures were carried out at room temperature. The GLI1 protein was separated on SDS-PAGE gels, transferred onto a nitrocellulose membrane and incubated in PBST buffer with 5% milk for 30 min. The membrane was washed with PBST buffer, incubated with polyclonal rabbit GLI1 antibody (Cell Signaling Technology; cat. no. 2354) (1:15,000 dilution) in PBST buffer with 5% milk for overnight at 4° C. The membranes were washed with PBST buffer (1×PBS, 0.3% Tween-20) and incubated with secondary antibody conjugated with HRP (Donkey anti Rabbit IgG-HRP, Santa Cruz Biotech; cat. no. sc-2077) for 1 hr (1:15,000 dilution in PBST with 5% milk). The membrane was then washed 3× with PBST buffer. The GLI1 protein was visualized using SuperSignal West Femto chemiluminescence kit (Thermo Scientific). For GAPDH western blot analysis, the same membrane was stripped with stripping buffer (Thermo Fisher) for 30 min. at room temperature and then incubated in PBST buffer with 5% milk for 30 min. The membrane was washed with PBST buffer, incubated with polyclonal rabbit GAPDH antibody (Cell Signaling Technology; cat. no. 14C10) (1:30,000 dilution) in PBST buffer with 5% milk for 1 hour at room temperature. The membranes were then washed with PBST buffer and incubated with secondary antibody conjugated with HRP (Donkey anti Rabbit IgG-HRP, Santa Cruz Biotech; cat. no. sc-2077) for 1 hr (1:30,000 dilution in PBST with 5% milk) at room temperature. The membrane was then washed 3× with PBST buffer. The GAPDH protein was visualized using SuperSignal West Pico chemiluminescence kit (Thermo Scientific).

Spontaneous Differentiation

Single cells were plated onto non-attachment plates and maintained in ESC media without FGF-β in the presence of FBS.

Spontaneous Differentiation in the Presence of GANT-61

WT H1 hESC single cells were plated onto non-attachment plates and maintained in ESC media without FGF-β in the presence of FBS. Experimental group was cultured in the presence of GANT-61 (Tocris Biosciences, Cat. No. 3191) at concentration of 5 μM, while the control group was cultured in the presence of 5 μM DMSO. Media containing GANT-61 was replaced every other day. Embryoid bodies were collected at days 10 and 20. Markers for the three embryologic lineages were analyzed by real time PCR.

Endodermal Differentiation

Endodermal differentiation was achieved essentially as described in 34. 10×10⁶ of pluripotent stem cells were plated on 60 mm Matrigel coated plates in advanced DMEM/12 supplemented with of 3 μM CHIR99021 (StemCell Technologies). Differentiation medium was changed every day. The cells were collected at day 3 and day 5 of differentiation.

Endothelial Differentiation

Endothelial differentiation was established by a monolayer induction protocol. Briefly, as described in 35, single cells were plated on 60 mm culture dishes coated with matrigel and cultured overnight in StemMACS iPS-Brew XF (Miltenyi Biotec). Differentiation was induced with an induction media containing advanced DMEM/12 (Life Technologies), glutamax (2.5 mM), ascorbic acid (60 μg/mL), and CHIR990921 (6 μM) added on day 0. On day 2 of induction, CHIR990921 was removed from the media. The cells were collected on day 5 of differentiation.

Hematopoietic Colony Forming Assay

The hematopoietic colony forming assay was performed in MethoCult H4435 medium (Stem Cell Technologies) supplemented with Flt-3L (50 ng/mL), IL-7 (20 ng/mL), IL-3 (5 ng/mL), SCF (50 ng/mL) and TPO (40 ng/mL) (Peprotech). After initial co-culture with OP9 mouse stromal cells as described in³⁶, hematopoietic progenitors were isolated on day 8 of differentiation and plated at density of 10×10⁵ cells per 35 mm dish. The colonies were evaluated after 16 days in culture.

Blast Colony Forming Assay

The blast colony assay was performed in MethoCult H4100 media mixed with SFEM (Stem Cell Tech) and supplemented with Heparin, LiCl, Glutamax MTG, Ascorbic Acid (all from Sigma-Aldrich), ExCyte (Millipore), FGF2, VEGF (Peprotech) and BIT 9500 Serum Substitute (Stem Cell Tech).

Neural Differentiation

Neural differentiation was performed using the PSC Neural Induction Medium (Thermo Fisher Scientific) according to manufacturer's instructions. Neural Progenitor cells (NPCs) were collected at day 7 of differentiation for early neural and at day 21 for late neural differentiation analysis.

Osteogenic Differentiation

Osteogenic differentiation was initiated using the MSCgo™ Osteogenic Differentiation Medium (Biological Industries) according to manufacturer's instructions.

Mineralization Assay

Matrix mineralization was quantified using alizarin red staining essentially as in ³⁷. After 3 min, cells were thoroughly washed and then de-stained with cetylpyridinium chloride. Absorbance of the de-stain solution was quantified using a ClARIOstar plate reader at A₅₃₈ₙₘ (BMG Labtech).

MTS Assay

MTS cell viability assay was performed using the CellTiter 96AQueous MTS (Promega, Madison, Wis.). Live cells were incubated in DMEM containing 10% FBS without the phenol red in the presence of MTS reagent for 1 hour. Absorbance was quantified using a ClARIOstar plate reader at MTS₄₉₀ₙₘ (BMG Labtech).

Flow Cytometry Analysis

Cells were harvested with StemPro Accutase (Thermo Fisher Scientific), washed with ice-cold FACS buffer (PBS+1% FBS+2 mM EDTA), and incubated with conjugated antibodies CD31 PE, CD34 FITC, VE-Cadherin APC (Miltenyi Biotech) for 30 min. at 4° C. Following this, cells were washed with a 0.5% BSA/PBS solution. Data collection was performed via the FACSCalibur (BD Biosciences) and analyzed with FlowJo software (version 10.5.3).

Immunohistochemistry

The following procedures were performed at room temperature. Neural cells were fixed with 3.2% paraformaldehyde for 30 min. and permeabilized for 5 min. with 0.1% Triton-x-100 in PBS. Cells were then treated with Dako Protein Block for 25 min. to prevent nonspecific antibody binding. Following this, neural cells were incubated with MAP2 (Santa Cruz, cat. no. SC-74421) and GFAP (Sigma, cat. no. G3893) mouse anti-human, primary antibodies. After washing the cells 3× with Dako Washing Buffer (WB), appropriate Alexa Fluor-conjugated secondary antibodies (Invitrogen) were added to cell culture wells; incubation time was 45 min. All antibody dilutions were performed according to manufacturers' instructions. Samples were then washed once more with WB and incubated with DAPI (Sigma Aldrich) for 3 min. The immunofluorescent cells were visualized with Leica DM IRB inverted microscope system (Leica, Germany) equipped with the Retiga 4000R camera (Qlmaging, Canada), which was controlled with Openlab software version 5.0.2 (Perkin-Elmer).

RNA Isolation

Total RNA was extracted with the RNeasy Mini Kit (Qiagen) via the instructions provided in the manufacturer's protocol. RNA quality and concentration were assessed with a Nanodrop instrument.

RNA Sequencing Analysis

Aliquots of RNA were submitted to Northwestern University's NUSeq Core. The mRNA library was prepared and the samples were analyzed using HiSeq 4000 Sequencing 50 bp, Single Reads. The list of differentially expressed genes was further analyzed using MetaCore and R Studio software (gplots and EnhancedVolcano packages).

Quantitative Real Time PCR

High-Capacity RNA-to-cDNA kit (Applied Biosystems) was used to reverse transcribe the isolated RNA. Each reaction tube included up to 2 g of RNA. The reverse transcription reaction was performed according to manufacturer instructions via the MBS Satellite (0.2 G) Thermal Cycler (ThermoFisher Scientific). The qPCR reaction mix was prepared by adding 12 ng of cDNA from each sample to the PowerUp SYBR Green Master Mix (2×) (Applied Biosystems). qPCR was performed (Standard Cycling Mode, primer Tₘ<60° C.) via the 7500 Fast Real-Time PCR system [Applied Biosystems]. The 7500 v2.3 software was used for data collection and gene expression comparisons (2^{-ΔΔCT} method). Primer sequences provided in Table 1.

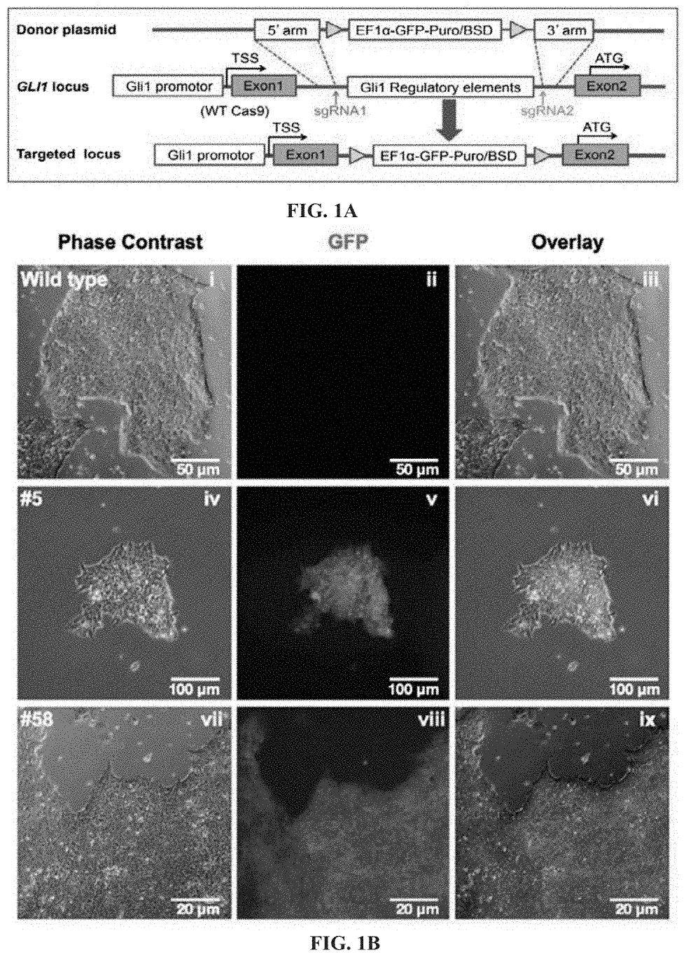

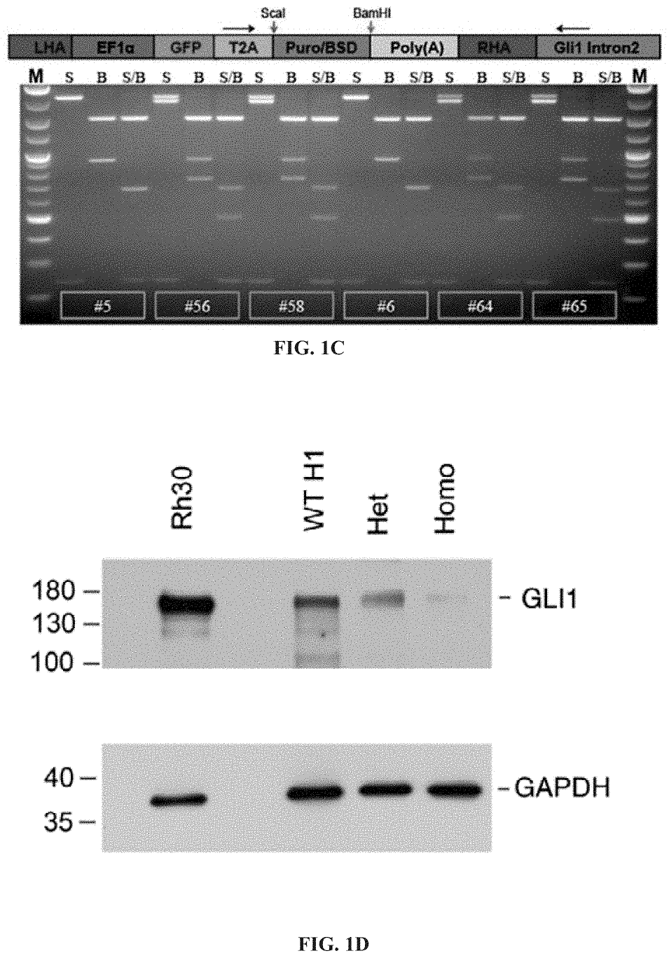

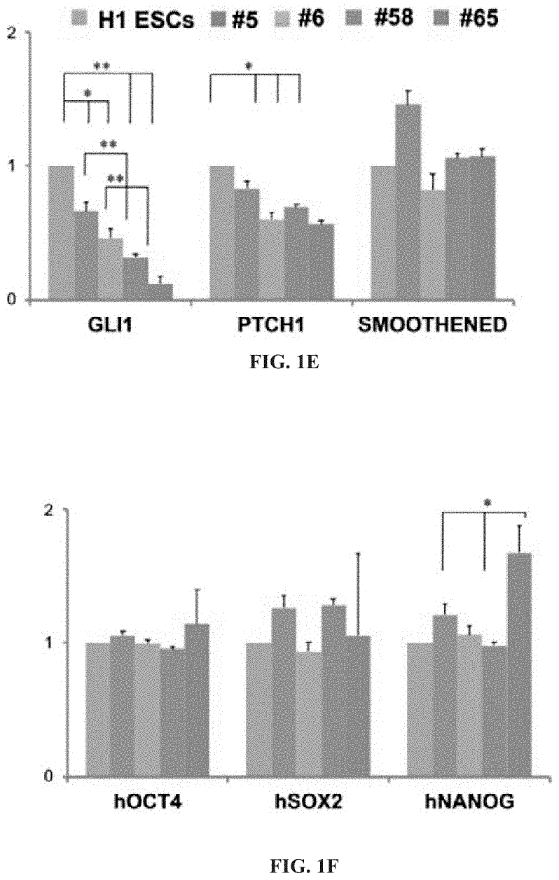

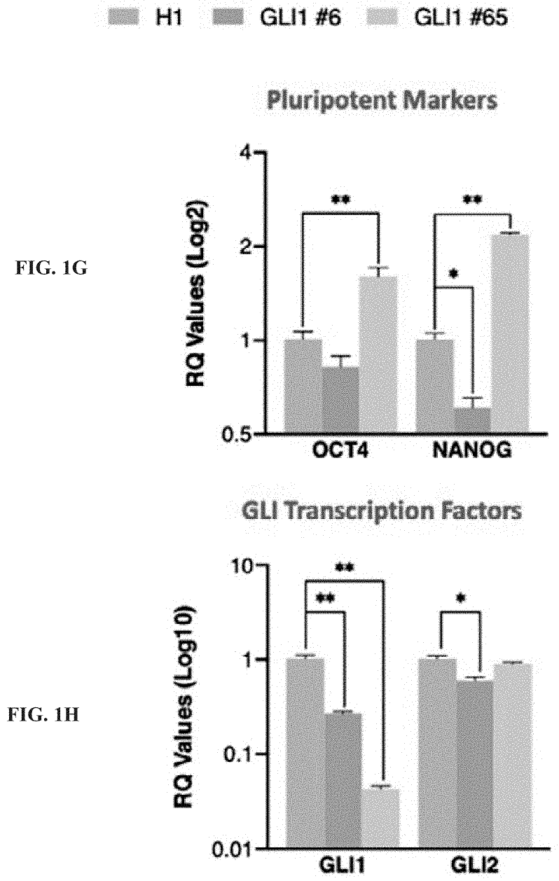

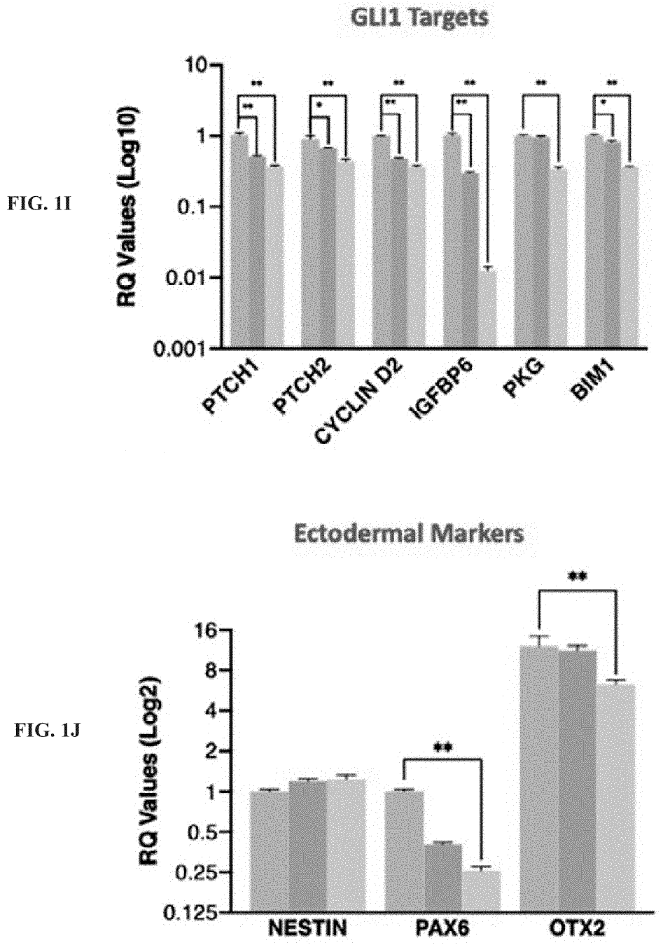

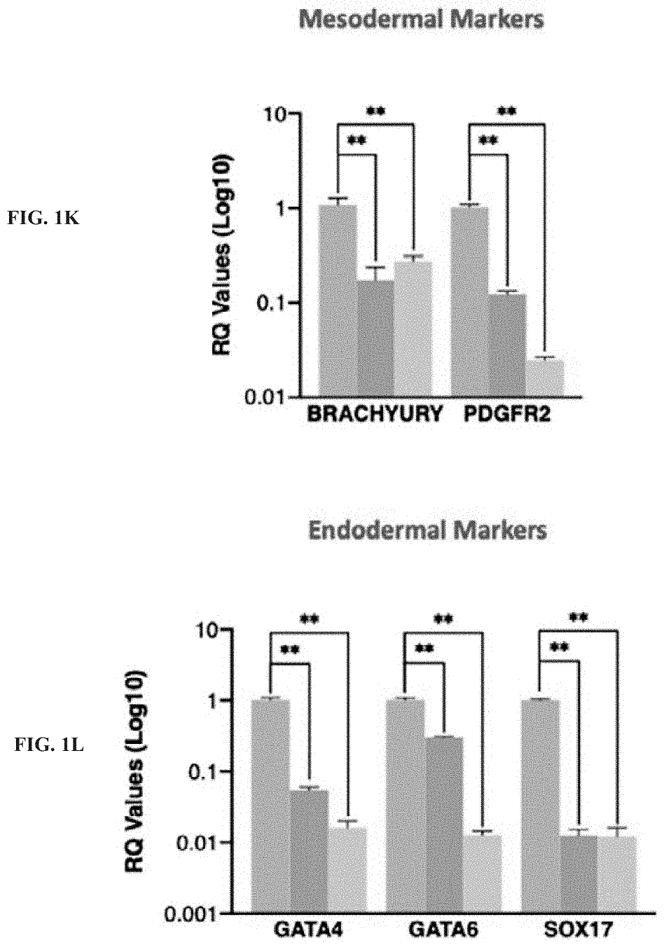

TABLE 1
CRISPR/Cas9 Sequences (Related to FIG. 1)

Upstream SgRNA: [symbol] (SEQ ID NO: 11)
5′-CACCGAGGGAGACCCCTACCGGGGC-3′ (SEQ ID NO: 12)
3′-CTCCCTCTGGGGATGGCCCCGCAAA-5′ (SEQ ID NO: 13)
Downstream SgRNA: TCACTCAGCGAGTGATGCTT (SEQ ID NO: 14)
5′-CACCGAAGCATCACTCGCTGAGTGA-3′ (SEQ ID NO: 15)
3′-CTTCGTAGTGAGCGACTCACTCAAA-5′ (SEQ ID NO: 16)
Upstream Sequence (210 bps, SEQ ID NO: 17)
ACTCCTGAGTCCCAAGGGCTGTGGGCAAGGAGCTCAGGAGGAGCCGGGGAG
ACCTTGTCTTGACCCTCTGACCTCAGGACCACCGGGGCAGCGGGAGCCAGCCGCAG
GGAGACCCCTACCGGGGCTGGGCGGGACCACTGGCCACTGCCAGCCTGTGTATCC
CCGTTGGCACCCCGCCCAAACGGGAGCTGGGGATCGAGGCCCCTCCTC
Downstream Sequence (222 bps, SEQ ID NO: 18)
CTCCCACCCAGGCAAAGCTCCCACCCAGTTCCCAAGAAGATCCCCAGAGTAC
ACAGACTACAAGACTGCCTCTGCCTCTCTGGGACATCATTTCCCCTTACCCCTCCCCT
CACTCAGCGAGTGATGCTTTTTTTGTTTTGAGACGGAGTCTAGCTCTGTCACCCAG
GCTGGAGTGCAGTGGCACCATCTCGGCTCACTGAAACCTCCGCCTCCCAGGTTCAA
Upstream Homolog Sequence (761 bps, SEQ ID NO: 19)
GCCCAGACAGAGGTGAGAAGGGGGGGCAGGCGGGGGACCACCTGGGAGCA
GTGGGGGAGGGGGCCTGAGGGGATGCTCAGCTTCTTAGGGACTCATCCCAGACCCG
GGACATAGAGGCAAAATAGGGGTGGGAGAGCCTGGGGTGAGACATTAGAAACTCC
AGATTTTTCACTTGTGTCTTTCTCTGTATCTTCTTTTTCTTCCCTTTTTTTCTTCTGTCA
GTCTGTGTATCTCTGTCTCAGGGAACCGTGGGTCTTTGTCTCCGCCTCTCCCATATAT
TAGAAATATCTTACTTTCATGCGGTTAAGTTTAAGAGGCTGGAGGGATGGCTAGCTG
GAGGTCTGCGTTGTAGAGAGGTAACCCCAGGTGTGTGTCTGCGCGTGGGGTAGGAA
GATGTCAGTGTTTCTGAAAGGTGGGGACTGCAAAGGAGGGAGCTCCAGGTGGGGTG
GGGACGGGTGTGTGGGAGGCAACAGAGCCACTAGGGGCCAGCCAGGCTTGAACCTT
TGACCTGTCTTGTGACAGATGTGCCAGTGGATGCTTGTGCTTTAGGGGAAAGGAGTG
TCTTCTGGACTTGGAAGGGGGCTGGGGCGGGGGGGGGCTGTCCAAGGTCTAGTGAA
GGCCCTAGAATGACCCCATGCAATTTGGACTCCTGAGTCCCAAGGGCTGTGGGCAA
GGAGCTCAGGAGGAGCCGGGGAGACCTTGTCTTGACCCTCTGACCTCAGGACCACC
GGGGCAGCGGGAGCCAGCCGCAGGGAGACCCCTAC
Downstream Homolog Sequence (801 bps, SEQ ID NO: 20)
GAGACGGAGTCTAGCTCTGTCACCCAGGCTGGAGTGCAGTGGCACCATCTCG
GCTCACTGAAACCTCCGCCTCCCAGGTTCAAGCGATTCTTCTGCCTCAGCCTTCCGA
GTAGCTGGGATTACAGGCACCCGCCATCATGACTGGCTAATTTTTGTTTTTTTGTAGA
GACGGGGGTTTCACCATGTTGGCCAGGCTGGTCTTCAACTGACCTCAGGTGATCCTC
CCGCCTCAGCCTCTCAAAGCGTTGGAATTACAGGCGTGAGCCACTGTGCCCGGCTCA
GTGATGCTCTTTTCAACTCGAATTCCGTGGCAGATGTCTTAGAGGGGTGGGGGATAC
CAGGGATGTTCTGCCCAGGATTCTGTGCCTGAGACTGCTGTCTGACAGTCTCTATTTC
CTCCACCTTTATACCTACCTTCCCTTTCTGCAGTGTCCCCACACCCTCCTCTGAGACG
CCATGTTCAACTCGATGACCCCACCACCAATCAGTAGCTATGGCGAGCCCTGCTGTC
TCCGGCCCCTCCCCAGTCAGGGGGCCCCCAGTGTGGGGACAGAAGGTCAGTGTATA
TACCAATCCCTGGGCCTTGAGGATTTGGCAGATCTCCCACTTGGGCCCACCCCCACC
CCATGCCAGTTTCCTATCTACAGGAGGATTTGAGGCCCCATGTCATATGGACCTGGA
ATCTGGGATCAGGTCATGTCTGGGGTTTTAAGGTGAGGTTTATGTATCCTCCATTCCC
ATTCCAGCTGTCTCTTTTTTCTAGGACTGTCTGGCCCGCCCTTCTGCCACCAAGCTAA
CCTC
Sequence of Blasticidin Gene (423 bps, SEQ ID NO: 21)
ATGAAAACCTTCAACATCTCTCAGCAGGATCTGGAGCTGGTGGAGGTCGCCA
CTGAGAAGATCACCATGCTCTATGAGGACAACAAGCACCATGTCGGGGCGGCCATC
AGGACCAAGACTGGGGAGATCATCTCTGCTGTCCACATTGAAGCCTACATTGGCAG
GGTCACTGTCTGTGCTGAAGCCATTGCCATTGGGTCTGCTGTGAGCAACGGGCAGAA
GGACTTTGACACCATTGTGGCTGTCAGGCACCCCTACTCTGATGAGGTGGACAGATC
CATCAGGGTGGTCAGCCCCTGTGGCATGTGTAGAGAGCTGATCTCTGACTATGCTCC
TGACTGCTTTGTGCTCATTGAGATGAATGGCAAGCTGGTCAAAACCACCATTGAGGA
ACTCATCCCCCTCAAGTACACCAGGAACTAA

siRNA Treatment and Hematopoietic Colony Forming Assay

Three days prior to CHIR99021 induction, H1 hESC cells were plated onto 6-well plate and transfected with 2 siRNA constructs, 4 ug each (obtained from Dr. Beletsky) using Lipofectamine 3000 (Invitrogen, Thermo Scientific, Waltham, Mass., USA) according to manufacturer's instructions. Briefly, prior to treatment, Lipofectamine 3000/siRNA complexes were prepared in reduced serum medium, OptiMEM (Invitrogen, Thermo Scientific, Waltham, Mass., USA), at the recommended ratio. Cells were then treated overnight in iPS-Brew Medium (Multeyi Biotech, CA, USA). To maintain the desired effect of GLI1 down-regulation during differentiation, the cells were transfected the second time 1 day prior to CHIR99021 treatment. The differentiation was then performed as in “Endothelial Differentiation” methods section, followed by “Hematopoietic Colony Forming Assay” methods section. Scrambled siRNA construct (obtained from Dr. Beletsky) was used as control.

Results

Clone Selection and Characterization

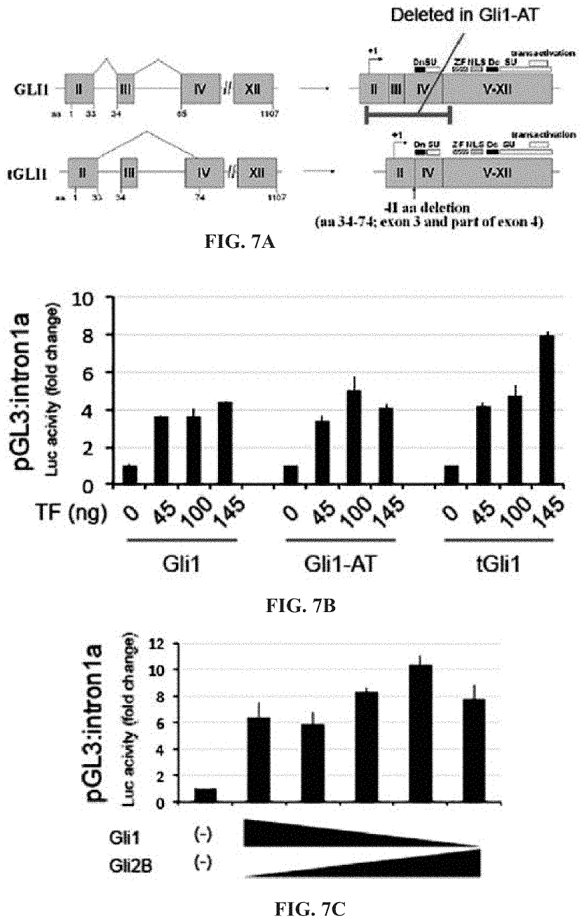

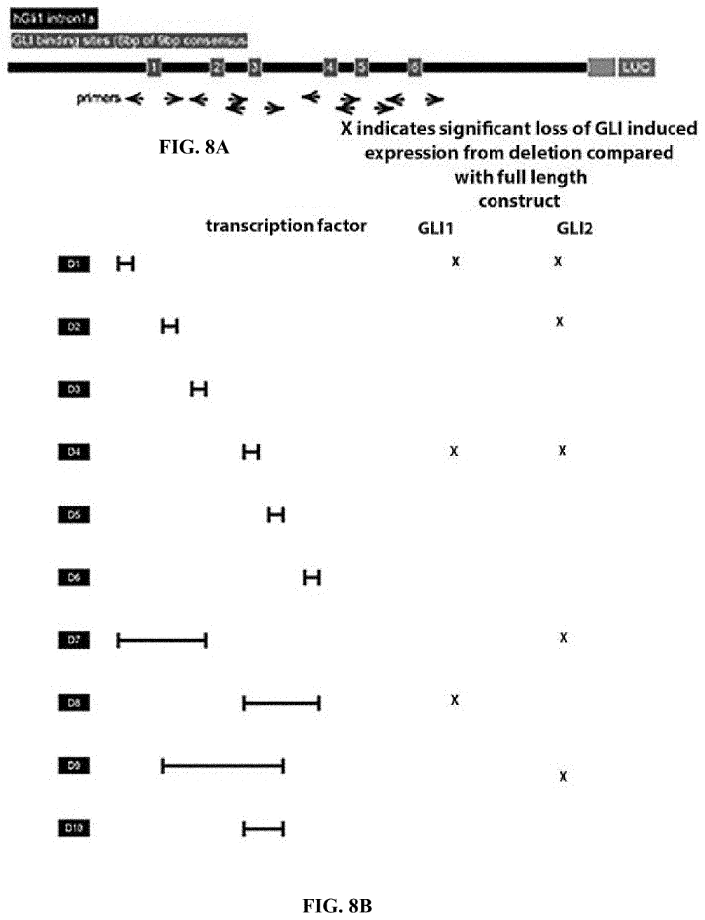

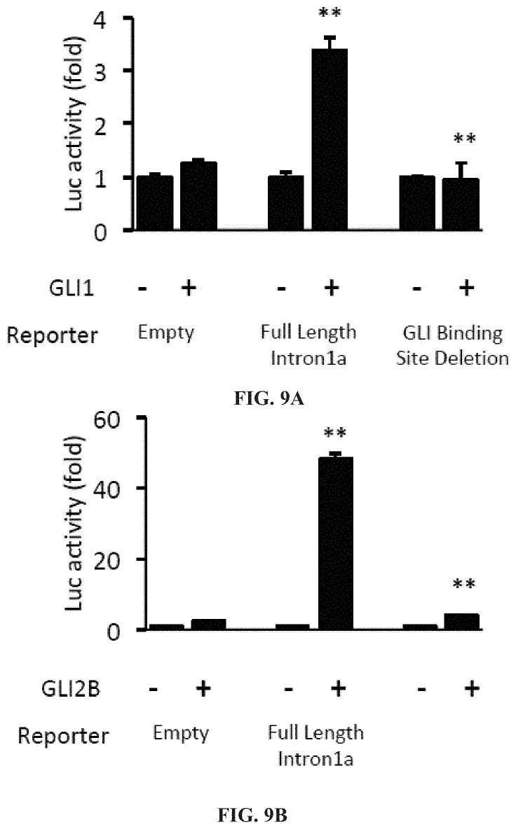

Previously, the first intron of the GLI1 gene has genomic characteristics of an enhancer and contains six conserved GLI binding sites (GBS) has been shown 2. The six GBS in the first intron of the hGLI1 gene bind GLI1 and GLI2 (Taylor, et al.) has been established. Further the binding events regulate gene expression. GLI1, GLI1-AT (GLI1 with a deletion removing the N-terminal repressor domain) and tGLI1 (an isoform of GLI1 present in tumors) all activate gene expression (FIGS. 7, 8). Moreover, there are no interactive effects of GLI1 or GLI2 on activation of gene expression (FIG. 8 and FIG. 9).

To determine the impact of this region on GLI1 expression in stem cells and stem cell differentiation, the region including the six GBS in the first intron of GLI1 gene were deleted using the CRISPR/Cas9 system (FIG. 1A). To increase selection specificity and to delete both alleles, two rounds of editing were performed using two different resistance markers. The final clones were picked manually based on their GFP expression (FIG. 1).

To validate successful CRISPR/Cas9 DNA modification, the isolated clones were genotyped (FIG. 1C) and sequenced (Table S1). Reduction of GLI1 expression was confirmed by Western blot analysis (FIG. 1D). Quantitative real time-PCR demonstrated that there was up to 50% reduction in GLI1 RNA in heterozygous clone (#6) compared to wild type clones while in homozygous clone (#65) GLI1 RNA was barely detectable. Furthermore, the results showed a significant decrease in the expression of the GLI1 target PTCH1 in heterozygous and homozygous clones (FIG. 1E). On the other hand, the deletion of this region had minimal effects on the expression of pluripotent markers OCT4, SOX2, and NANOG as shown by real time-PCR (FIG. 1F). These data indicate that GBS deletion, in the first intron of the GLI1 gene, dramatically reduces GLI1 expression in stem cells, without affecting their pluripotency.

Spontaneous Differentiation

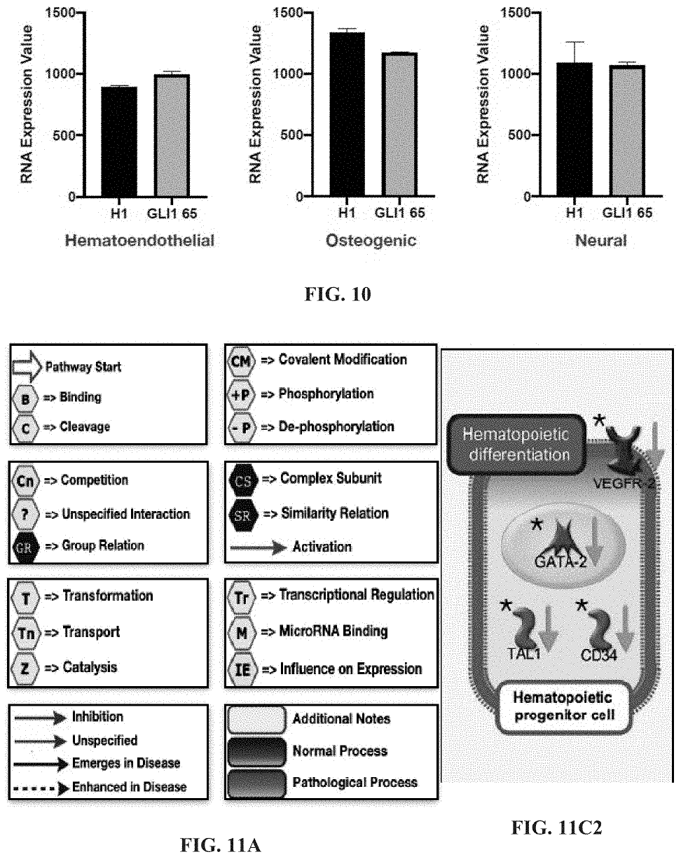

To examine the cellular effects of the deletion of the GBS region of the GLI1 gene, spontaneous differentiation of heterozygous and homozygous clones was conducted, with the most significant down-regulation of GLI1, and wild type pluripotent cells using the embryoid body method. Single cells were plated onto non-attachment plates and maintained in ESC media without FGF-3 in the presence of FBS. On day 10 of differentiation, embryoid bodies were collected and early markers of the three embryonic lineages were assessed by real time-PCR. The results showed that during spontaneous differentiation, the homozygous edited clone (#65) retained pluripotency markers at a higher level than the heterozygous clone (#6) or WT H1 hESC control (FIG. 1G) indicating the edited cells were held in a pluripotent state. The expression of GLI1 (FIG. 1H) and its targets (FIG. 1I) remained low. The analyzed markers: ectodermal PAX6 and OTX2 (FIG. 1J), mesodermal BRACHYURY and PDGFRα (FIG. 1K), and endodermal GATA4 and GATA6 (FIG. 1I) were significantly down-regulated for both heterozygous and homozygous GLI1 edited clones compared to the WT H1 hESC differentiated control. These data suggest that the deletion of the region containing the GBS significantly affects stem cell differentiation toward all three embryonic lineages, and this effect is most pronounced in the homozygous state. Furthermore, GLI2 expression remained consistent during spontaneous differentiation (FIG. 1H) and later during directed differentiation (FIG. 10), which shows that unlike in the case of the null GLI1 mouse model, GLI2 does not compensate for GLI1 after 6 GBS are deleted.

Directed Differentiation

To more precisely define the effect of GBS deletion on each lineage, directed differentiation experiments were conducted. The aim was to determine whether down-regulation of the early differentiation markers, observed during spontaneous differentiation, was due to delayed or inhibited differentiation potential or because the edited cells were differentiating faster than the WT H1 hESC control. Furthermore, the observed significant down-regulation of the genes was due to the effect of GLI1 editing was verified, rather than non-specific 3D interaction within the embryoid body differentiation system. The differentiated cells were assessed using functional assays and analyzed using RNA sequencing (RNA-Seq), real time-PCR, fluorescence-activated cell sorting (FACS) analysis, and immunofluorescence (IF). For directed differentiation experiments, the homozygous GLI1 edited clone with the most down-regulated GLI1 expression was chosen, clone (#65). Experiments performed with clone #65 showed that GLI1 down-regulation had the most significant effect on differentiation to mesodermal, specifically hematopoietic lineages. To confirm these results, the experiments using homozygous clone #58 were repeated, and performed siRNA treatments. Furthermore, to ensure that these data are specific to homozygous GLI1 down-regulation, directed differentiation toward hematopoietic lineages using heterozygous clone #6 was performed.

Early Mesodermal Potential of the GLI1 Edited Clone

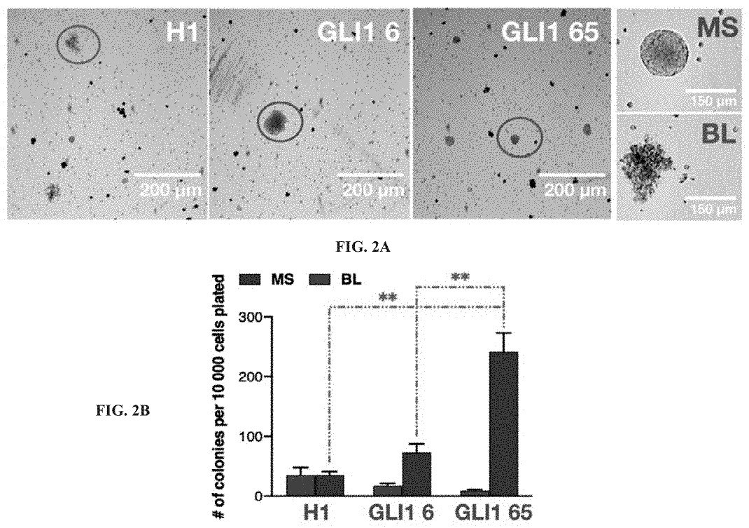

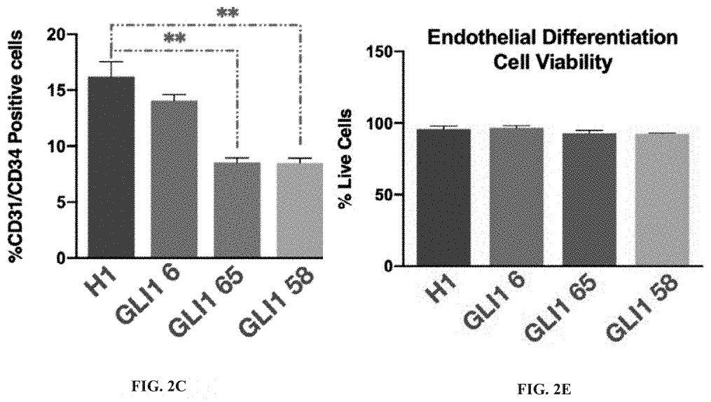

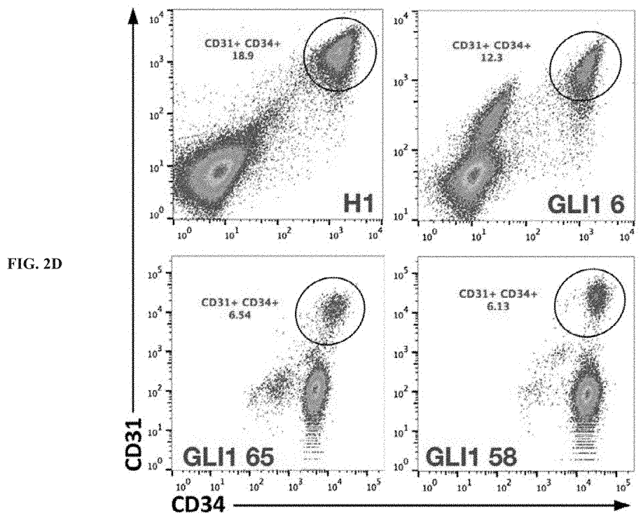

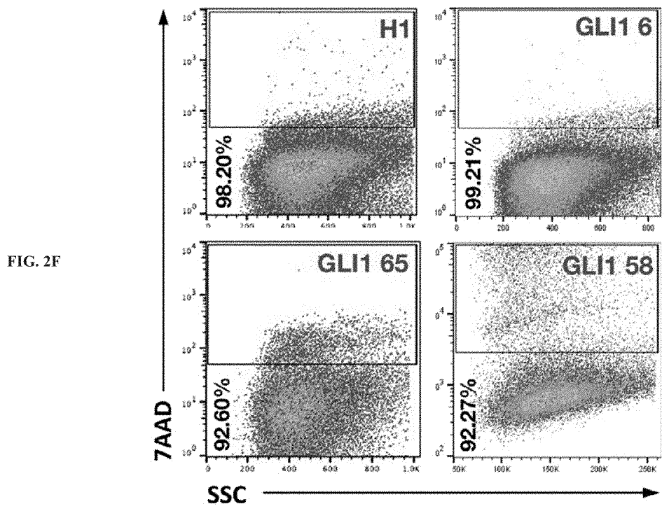

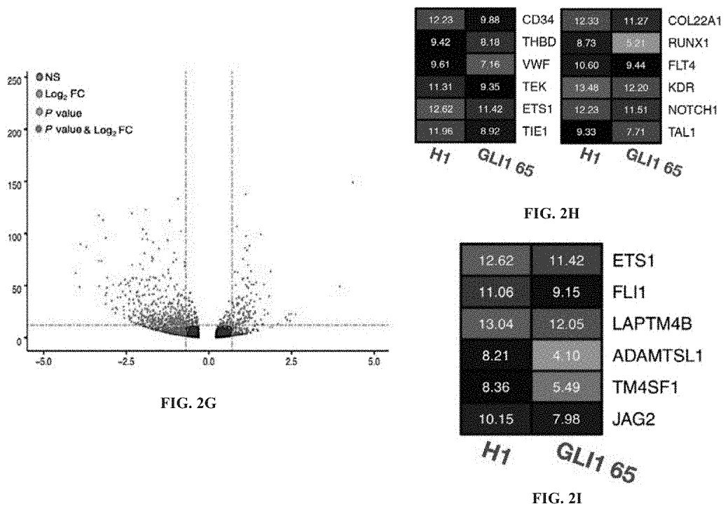

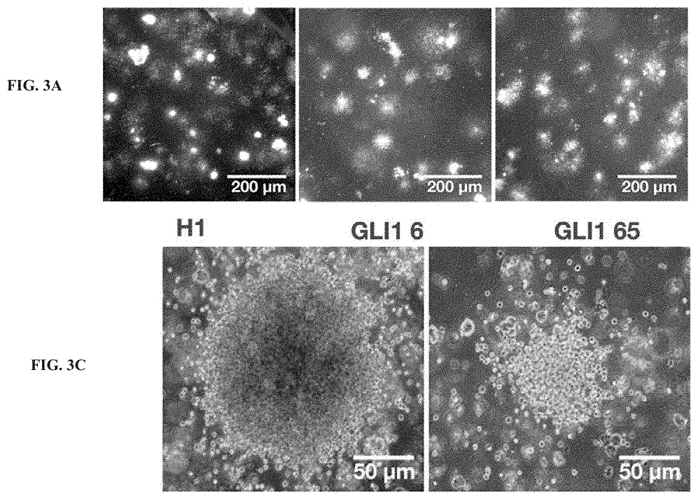

To detect early phenotypic differences in mesodermal differentiation between heterozygous clone #6, homozygous GLI1 edited clone #65, and WT H1 hESCs, differentiation was induced by co-culturing pluripotent cells with OP9 mouse stromal cells. On day 3 of co-culture, the colonies were separated into single cells and placed in semi-solid media. After 16 days in culture, the resulting mesenchymal (MS) and Blast (BL)-(precursors of primitive blood and endothelial cells) (FIG. 2A) colonies were counted by two individuals and the mean number of colonies was determined.

We found that an approximately equal number of MS and Blast colonies developed in the WT H1 hESCs. There was no significant difference between the number of colonies generated by WT H1 hESC and the heterozygous GLI1 edited clone #6. The homozygous GLI1 edited cells generated significantly more MS colonies than the heterozygous GLI1 edited clone #6 and the WT H1 hESCs (FIG. 2B). These data indicate that homozygous deletion of 6 GBS of GLI1 skews the early stage mesodermal commitment toward the mesenchymal component and results in a smaller number of blast colonies, indicating less prevalence for primitive blood and endothelial cells.

Endothelial Differentiation of GLI1 Edited Clones (Mesodermal Lineage)

To further evaluate the effects of GLI1 down-regulation on endothelial cell formation, endothelial differentiation was performed as previously described 38. Heterozygous GLI1 edited clone #6 were used and two homozygous GLI1 edited clones: #65 and #58 for this experiment. On day five of differentiation, the number of CD31+CD34+ endothelial progenitor cells were slightly reduced in the heterozygous GLI1 edited clone #6 compared to the WT H1 hESCs. The number of CD31+CD34+ endothelial progenitor cells in the homozygous GLI1 edited clones #65 and #58 was significantly reduced and on average 4 times smaller compared to the WT H1 hESCs (FIG. 2C and FIG. 2D). This showed that GLI1 down-regulation significantly inhibits endothelial development. The assessment of cell viability using 7-AAD dye showed that the percentage of live cells in WT and GLI1 edited differentiating cultures was very high, around 92-97% on average (FIG. 2E). There was no significant difference between the numbers of 7-AAD positive cells (FIG. 2F). This suggested that the low efficiency of endothelial differentiation was due to the inhibition of differentiation, rather than poor cell survival.

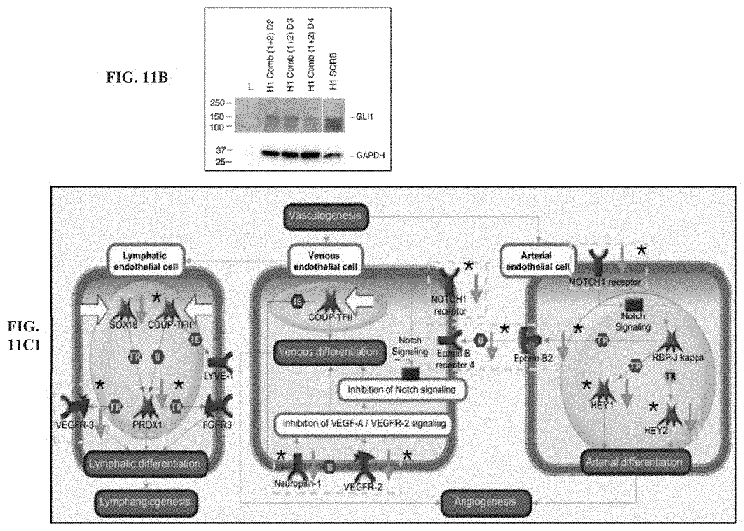

Analysis of RNA-Seq data, collected on day 5 of endothelial differentiation, confirmed these results. Using the volcano plot generated by R-Studio software (FIG. 2G), differentially expressed vascular and endothelial-related genes with the lowest p-value and highest fold change (FC) were selected. The selected gene set incorporated genes that are involved in promoting vascular homeostasis by regulating proliferation, migration, adhesion, actin cytoskeletal reorganization, and anti-inflammatory mechanisms in vascular tissue. In comparison to the WT H1 hESC control, this gene set was significantly down-regulated in the GLI1 edited cells: CD34 (p-value=3.22×10⁻²⁴⁹, FC=−2.32), THBD (7.90×10⁻⁵, −0.97), VWF (1.73×10⁻⁷⁰, −2.32), TIE1 (5.15×10⁻⁵¹, −2.72), TEK (2.37×10⁻¹²³, −1.92), ETS1 (5.62×10⁻¹¹³, −1.19), FLT4 (1.62×10⁻⁴, −0.91), KDR (2.29×10⁻³⁰, −1.23), NOTCH1 (8.85×10⁻⁴, −0.63), TAL1 (2.78×10⁻³⁰, −1.53) and COL22A1 (4.83×10⁻⁸², −1.05). Furthermore, the data showed significant down-regulation in the RUNX1 gene (2.68×10⁻⁵⁸, −3.08)—indicating decreased hemogenic potential of derived endothelium in GLI1 edited cells (FIG. 2H). Pathway analysis revealed significant inhibition of differentiation toward lymphatic, venous and arterial endothelium as indicated by significant down regulation in SOX18 (3.12×10⁻¹⁰, −1.39), PROX1 (1.73×10⁻⁷⁰, −2.32) and VEGFR-3 (9.16×10⁻⁴, −0.91), HEY1 (3.12×10⁻¹⁰, −0.66) and HEY2 (1.00×10⁻⁵⁰, −1.85) (FIG. 11A-C).

Additionally, several genes were observed, implicated in various forms of cancer were significantly down-regulated in the GLI1 edited cells during vascular formation. LAPTM4B (p-value=1.13×10⁻⁷⁵, −0.99) is related to tolerance to metabolic stress in cancer cells 39. ETS1 (5.62×10⁻¹¹³, −1.19) and FLI1 (5.37×10⁻⁹⁵, −1.86) are transcription factors in a family that have been associated with GLI1 up-egulation in Ewing sarcoma through the EWSR1/FLI1 translocation fusion protein⁴⁰. FLI1 up-regulation is also associated with susceptibility to follicular non-Hodgkin lymphoma. ADAMTSL1 (4.17×10⁻⁵¹, −3.32) has been associated with chondrosarcoma. ADAMTSL1 may play an important role in cell survival and its down-regulation has been linked to significant anti-tumor effects⁴¹. TM4SF1 (2.30×10⁻⁴¹, −2.53) is highly expressed in various carcinomas. JAG2 (4.15×10⁻⁷¹, −2.08), was found to be overexpressed in multiple myeloma, but not in nonmalignant plasma cells from tonsils or bone marrow from healthy individuals, or patients with other malignancies⁴² (FIG. 2I). These findings may suggest that the possible role of these genes in these cancers is related to tumor angiogenesis.

Hematopoietic Potential

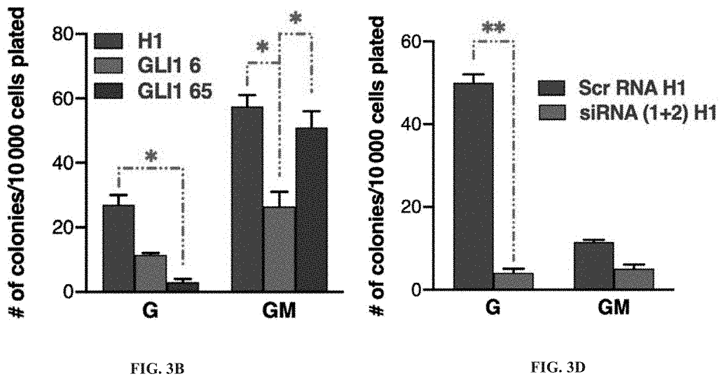

GLI1 down-regulation has been shown to affect differentiation and proliferation of myeloid progenitors in mice⁴¹. To assess the hematopoietic potential of heterozygous GLI1 edited clone #6 and the homozygous GLI1 edited clone #65, co-cultured the cells isolated on day 5 of endothelial differentiation with OP9 mouse stroma. Hematopoietic progenitors were then placed into semi-solid media containing hematopoietic cytokines. After 18 days, the colonies were counted and the numbers were compared to WT H1 hESCs (FIG. 3A). No significant difference in the number of granulocyte forming units (CFU-G) between WT H1 hESCs and heterozygous GLI1 edited clone #6 were observed. The difference in the number of CFU-G between the WT H1 hESCs and homozygous GLI1 edited clone #65 was significant. On average, the WT H1 CFU-G count was 9× greater than the edited GLI1 clone CFU-G count (FIG. 3B).

To confirm that the observed result was due to GLI1 down-regulation, WT hESC H1 were treated with a combination of two siRNAs. GLI1 down-regulation was confirmed using Western Blot (FIG. 11B). Scrambled RNA treated progenitors on average formed 12.5× greater CFU-G (FIG. 3C) and those colonies were marginally smaller in size (FIG. 3D).

GLI1 siRNA Sequences (Related to FIG. 3)

| Name | Sequence ID Nos. |
|---|---|
| ID No. 1 | 5′- GAAGCGUGAGCCUGAAUCUGUdTdT- 3′ |
| ID No. 2 | 5′-ACAGAUUCAGGCUCACGCUUCdTdT 3′ |
| ID No. 3 | 5′- GCCCUUCAAAGCCCAGUACAUdTdT- 3′ |
| ID No. 4 | 5′-AUGUACUGGGCUUUGAAGGGCdTdT 3 ′ |
| ID No. 5 | 5′- GGCUGCACCAAACGCUAUACAdTdT- 3′ |
| ID No. 6 | 5′-UGUAUAGCGUUUGGUGCAGCCdTdT 3′ |
| ID No. 7 | 5′ UCUGUAUAGCGUUUGGUGCAGCdTdT 3′ |
| ID No. 8 | 5′ GCUGCACCAAACGCUAUACAGAdTdT 3′ |
| ID No. 9 | 5′ AAGAUUGGCGGUGUUCCUUUGCdTdT 3′ |
| ID No. 10 | 5′ GCAAAGGAACACCGCCAAUCUUdTdT3′ |

Osteogenic Differentiation

GLI1 has been implicated in SHH-mediated specification of the osteoblast lineage⁴⁴. Previous work established that GLI1 expressing mesenchymal progenitor cells are responsible for bone formation and fracture repair in mice⁴⁵. The differentiation potential of the MSCs differentiated from the WT H1 hESCs and the homozygous GLI1 edited cells. Mesenchymal differentiation was established by isolation of a multipotential progenitor at the mesenchymoangioblast stage as described previously⁴⁶.

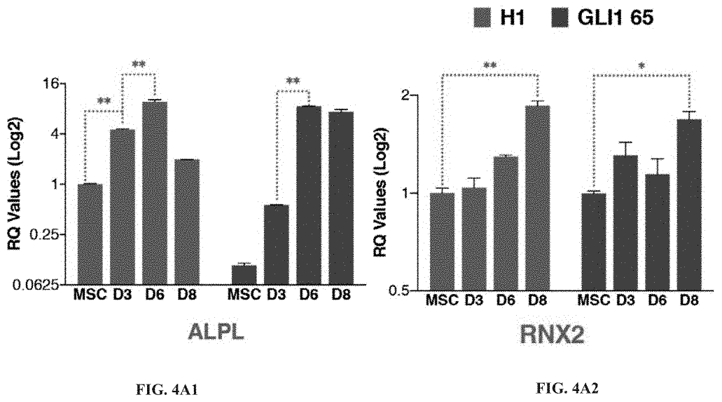

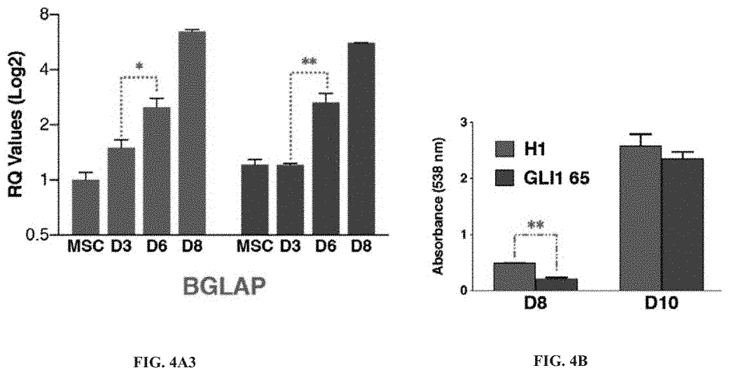

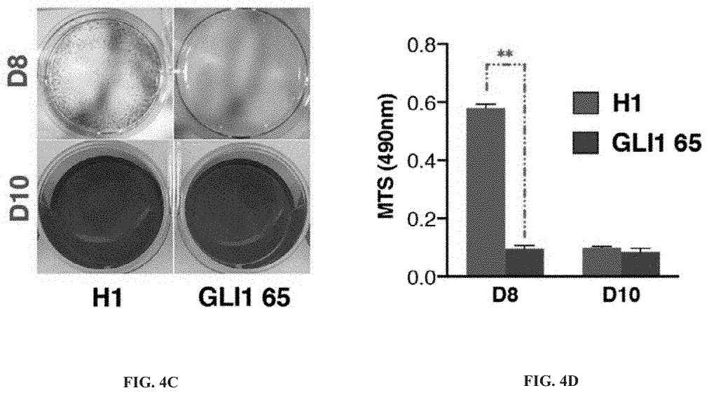

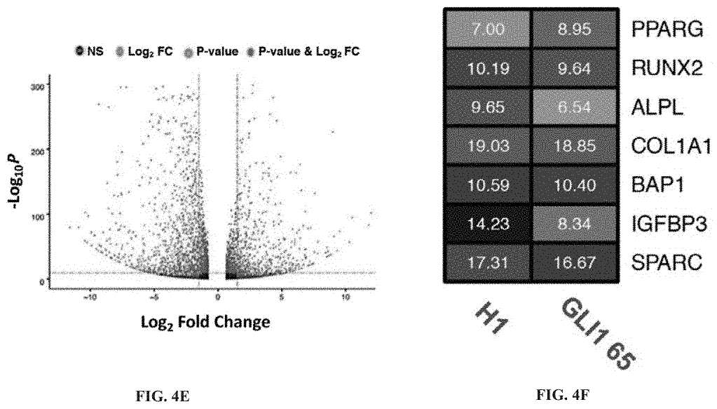

Our time point real time PCR analysis of osteogenic differentiation showed that ALPL, an early marker of osteogenic differentiation, increased significantly in WT H1 hESCs by day 3. Whereas in the homozygous GLI1 edited cells, the significant increase occurred between day 3 and 6. By day 6, there was no significant difference in ALPL gene expression between homozygous cells and WT control. The real time PCR results also showed no significant difference between the expression of other osteogenic markers, including RUNX2 and BGLAP (FIG. 4A). These data suggest that the loss of GLI1 may delay the initiation of bone development.

Evaluation of osteogenic differentiation efficiency using Alizarin Red staining complements the real time PCR data. At day 8, the results demonstrated that WT H1 hESC1 had a significantly greater amount of calcium deposition. By day 10 of differentiation, both WT H1 hESCs and homozygous GLI1 edited cells were highly mineralized (FIG. 4B, C). There was no significant difference between absorbance values at 538 nm.

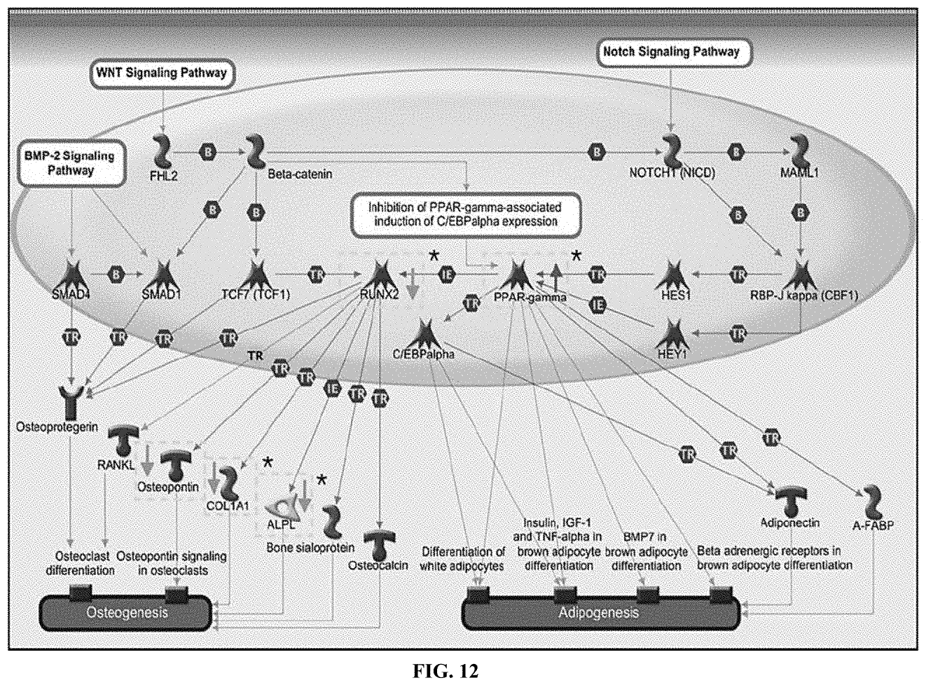

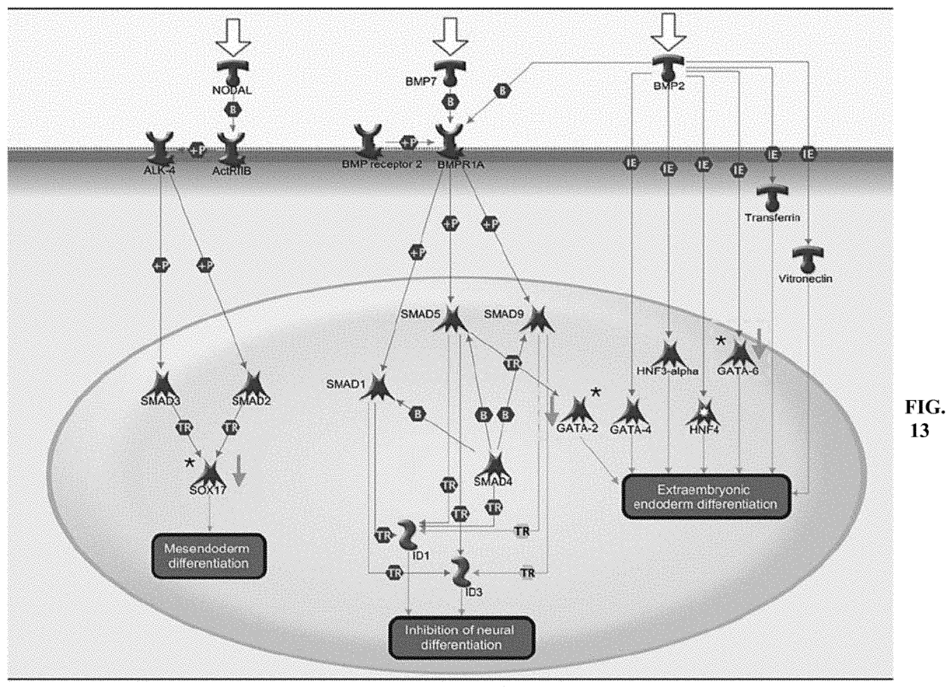

Additionally, the MTS viability assay revealed that the initiation of osteogenic differentiation greatly affected viability with homozygous editing. On day 8 of differentiation, the homozygous clone had 5 times fewer proliferating cells than the WT H1 hESC. The difference became insignificant as differentiation progressed and the number of viable cells decreased in WT control (FIG. 4D). This proliferative profile is reflected in SHH pathway signaling, which promotes osteogenic induction. In comparison to WT H1 hESCs, the GLI1 edited cells had up-regulated expression levels of the transcription factor PPAR-gamma (PPARG, p-value=2.20×10⁻³⁷, FC=1.94), which inhibits the transcription factor RUNX2 (8.73×10⁻¹¹, −0.55). Down-regulated RUNX2 expression cannot effectively up-regulate key osteogenesis-promoting genes such as ALPL (3.78×10⁻¹⁵, −2.99) and COL1A1 (1.77×10⁻⁵, −0.18) (FIG. 12).

Furthermore, additional pathway analysis and the volcano plot (FIG. 4E) showed an even larger array of genes that were deferentially expressed with high significance. In addition to PPARG, RUNX2, ALPL, and COL1A1, the osteogenic gene list was expanded to include, BAP1 (p-value=0.01, FC=−0.19), IGFBP3 (0, −5.86), and SPARC (1.30×10⁻⁴⁷, −0.64. These genes were significantly down-regulated in the homozygous cells (FIG. 3F).

Endodermal Potential (Endodermal Lineage)

Endodermal differentiation was achieved using a monolayer differentiation system as previously described with addition of CHIR99021 (3 uM)³⁴. To assess the dynamic of differentiation, the samples were evaluated at day 3 by real time PCR and at day 5 by RNA-Seq.

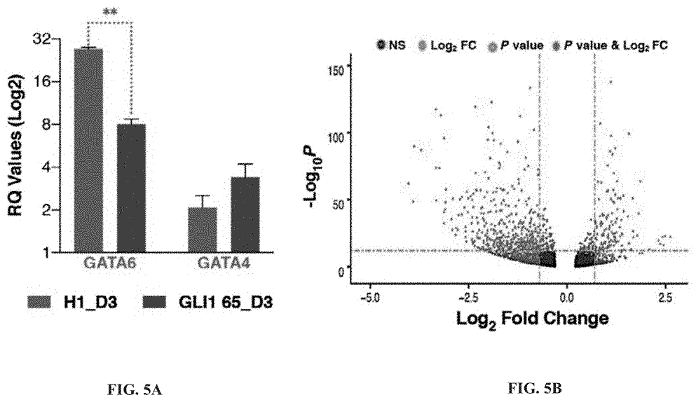

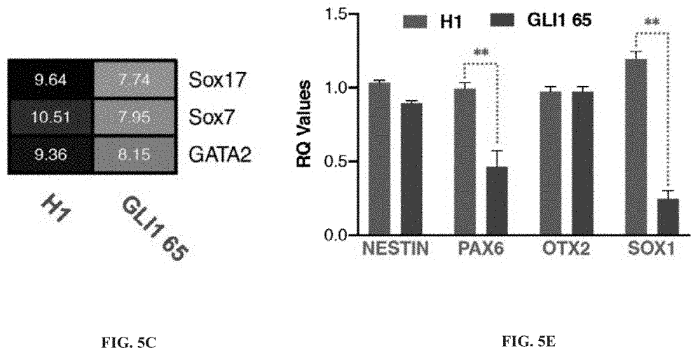

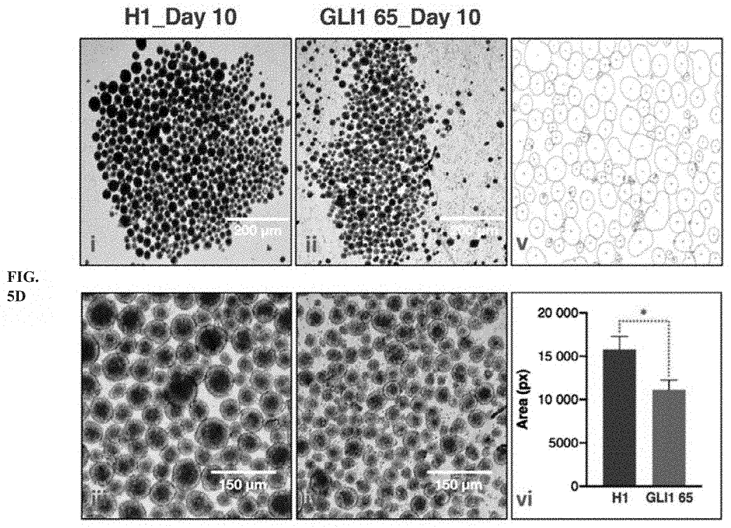

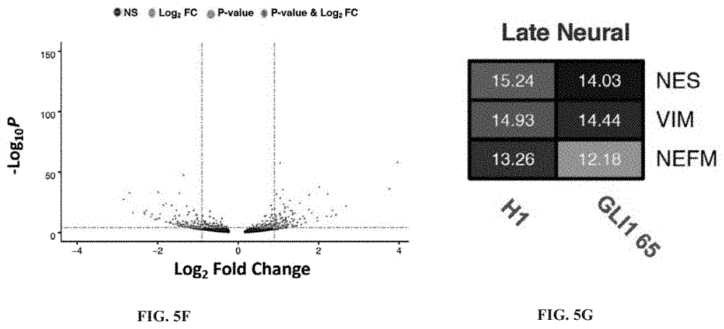

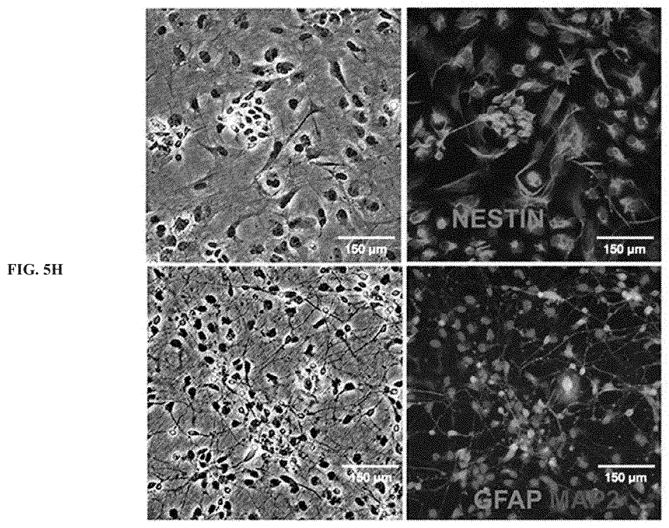

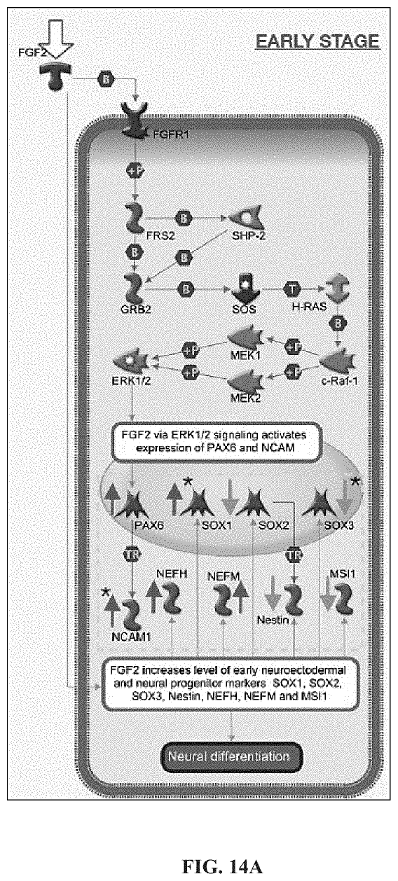

As with the spontaneous differentiation result, real time PCR results and RNA-Seq data demonstrated a significant down-regulation of GATA-6 in GLI1 edited cells as compared to WT H1 hESCs (FIG. 5A). From the volcano plot and pathway analysis (FIG. 5B and FIG. 13), SOX17 was observed at (p-value=2.12×10⁻¹², FC=−1.57), SOX7 (1.94×10⁻³³, −2.27) and GATA-2 (6.32×10⁻²², −1.15) were expressed at a significantly lower level in homozygous GLI1 edited cells compared to WT hESCs (FIG. 5C).

Neural Differentiation of GLI1 Clones (Ectodermal Lineage)

The effects of GLI1 down-regulation on ectodermal differentiation were assessed using PSC neural induction medium. On day 7 of differentiation neural progenitor cells (NPCs) were collected and assessed using real time-PCR. Half of the cells were transferred to NPC culture media and propagated for 4 passages. To assess the morphological properties of NPCs, NPC's were cultured as neurospheres. The results showed that the homozygous GLI1 edited cells had a reduced ability to form neurospheres in that they were smaller in size and in number (FIG. 5D).

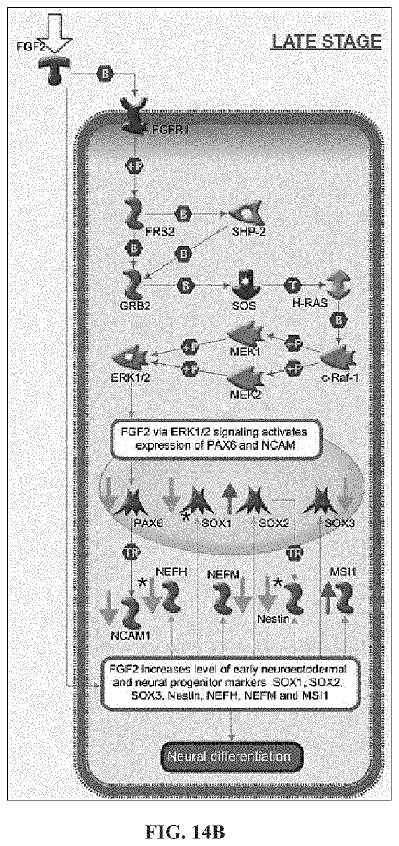

Our real time PCR analysis revealed that during the initial stages of differentiation, at day 7, there was a significant difference in expression of PAX6 and SOX1. Both genes were down-regulated in GLI1 edited cells (FIG. 5E), while NCAM1 was significantly up-regulated (FIG. 14A-14B). At day 28 of NPC culture, the volcano plot (FIG. 5F) and pathway analysis (FIG. 14B) showed a significant down-regulation of NES (1.13×10⁻⁴, −0.88), VIM (2.48×10⁻⁵, −0.47) and NEFM (1.19×10⁻⁹, −1.02) (FIG. 5G). This suggested that editing of GLI1 inhibited the expression of early neural genes.

To verify whether these early phenotypic differences had an adverse effect on maturation potential of NPCs derived from the GLI1 homozygous edited cells, spontaneous terminal neural differentiation was conducted. IF staining showed that homozygous NPC differentiated to neurons and glial cells, as indicated by positive MAP2 and GFAP expression (FIG. 5H).

Spontaneous Differentiation in the Presence of Gant-61

GANT-61 was found to inhibit GLI1 and GLI2 activity by inhibiting the binding of both GLI1 and GLI2 transcription factors to DNA⁴⁷. GANT-61 binds GLI1 (a 5-zinc finger protein) between zinc fingers 2 and 3 at sites E119 and E167. GANT-61 binds GLI1, but not all zinc finger transcription factors and it does not bind DNA⁴⁸. The effects of GANT-61 on differentiation using the stem cell model was evaluated.

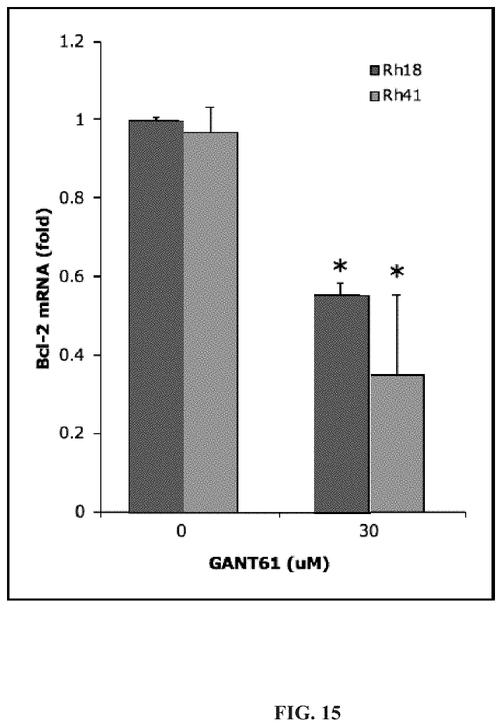

First, to validate the effect of GANT-61 inhibition on GLI1 target genes, rt-PCR on GANT-61 treated Rh18 and Rh41 cells was performed. These cell lines express GLI1. Significant down-regulation of Bcl-2 in both Rh18 and Rh41 cells (FIG. 15) was observed.

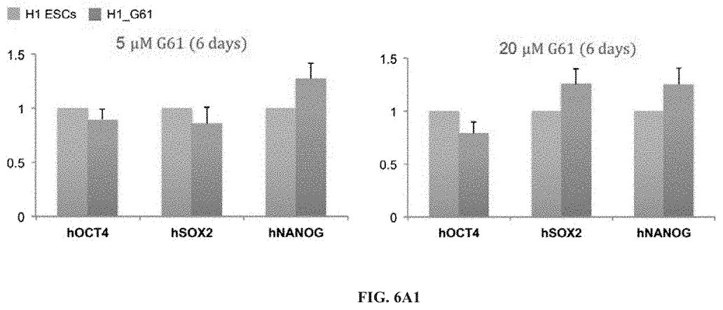

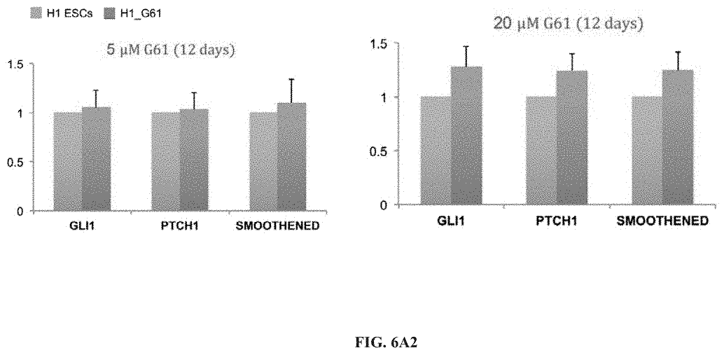

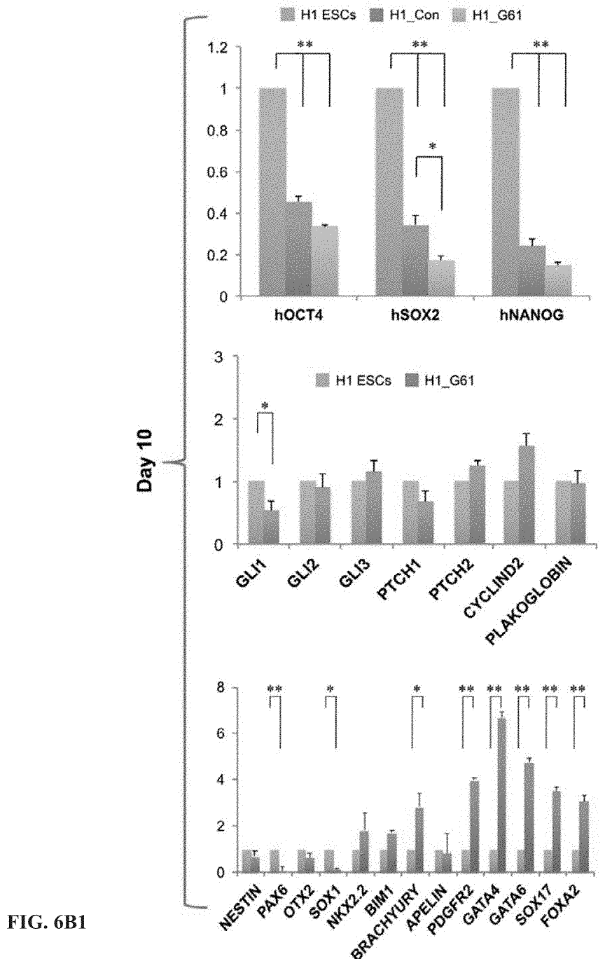

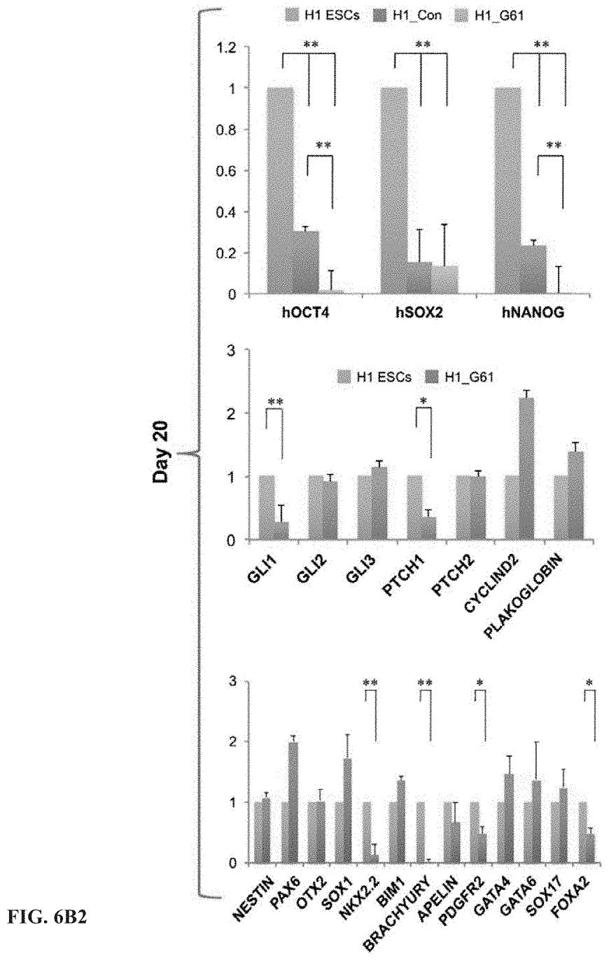

Next, the effect of GANT-61 on GLI1 and SHH pathway members PTCH1 and SMO expression and on pluripotency marker expression in hESCs was evaluated. There were no significant differences in expression of these markers following 6 or 12 days of GANT-61 treatment (FIG. 6A).

Following this, the effect of GANT-61 exposure during spontaneous differentiation was evaluated. After 10 days of differentiation treatment in the presence of GANT-61 mesodermal and endodermal gene expression significantly increased, while ectodermal gene expression decreased. After 20-days of exposure, GANT-61 significantly decreased endodermal and mesodermal marker expression (FIG. 6B). After 20-days of exposure to GANT-61, the effects were similar to those achieved with the GLI1 edited clones. GLI1 and its target PTCH1 were significantly down-regulated during the differentiation process (FIG. 6B).

Discussion

GLI1 expression is associated with about a third of human cancers. Its positive feedback loop with GLI2, normally quenched by PTCH1, can drive cancer growth and decrease cancer cell apoptosis^{10,21}. In previous work, developing a greater understanding of the regulatory region in the first intron of the GLI1 gene may provide critical insight into the mechanisms governing this feedback loop. The first intron not only contains highly conserved binding domains, but also acts as a transcriptional enhancer when stimulated by GLI transcription factors and is important in controlling cell proliferation, apoptosis, and differentiation²⁴.

Based on these findings, a human stem cell model was developed to study the effects of GLI1 expression during the earliest stages of human development. This was achieved by precise editing of a complex enhancer in the first intron of the human GLI1 gene²⁴. The result was a deletion of six highly conserved GLI binding sites (GBS) and down-regulation of GLI1 expression. The data show that significant reduction in GLI1 expression occurs in the heterozygous edited state, and that GLI1 expression is essentially eliminated when both alleles of the region are mutated (homozygous state). This strongly supports the notion that this region is responsible for the positive feedback on GLI1 expression by GLI1 and GLI2²⁴. Furthermore, elimination of this intronic region also significantly affects stem cell differentiation toward all three embryonic lineages. The data demonstrated that, GLI1 editing did not affect pluripotency marker expression, it did inhibit mesodermal and endodermal commitment, and it caused a significant gene dysregulation during neural (ectodermal) differentiation.

With regard to mesodermal commitment, a recent differential gene analysis of GLI1/GLI2 binding regions identified blood vessel development as one of the most up-regulated biological processes¹⁶. GLI1 editing significantly impacts vasculogenesis was found. During early stages of hemato-endothelial (mesodermal) differentiation, the GLI1 edited cells were skewed toward mesenchymal rather than endothelial and primitive blood progenitors. At later stages, formation of endothelium was significantly inhibited. Furthermore, expression of RUNX1, an endothelial marker with elevated expression levels in emergent hematopoietic progenitor cells⁴⁹ and during definitive hematopoiesis⁵⁰,⁵¹ was significantly down-regulated in GLI1 edited clones.

To complement these results, the number of CFU-G was also significantly decreased in edited clones. A similar result was observed after WT hESC H1 was treated with a combination of siRNAs aimed at GLI1 down-regulation. Interestingly, this phenomenon with human cells was also observed by⁴³, who found that GLI1^{null} mice have a decreased response to granulocyte colony stimulating factor (G-CSF) in vivo and a decreased myeloid development potential in vitro. The small molecule GANT-61 inhibited mesodermal commitment of H1 hESCs in embryoid bodies. Thus, it is reasonable to suggest that though GANT-61 is inhibiting tumor growth in part by inducing apoptosis, a major component of GANT-61 tumor suppression is its anti-angiogenic effect.

Studies in mice demonstrated that GLI1 can induce early osteoblast differentiation during bone formation 44. Furthermore, GLI1 regulates mature bone metabolism by promoting osteoblast differentiation and repressing osteoblast maturation toward osteocytes.⁵². The data and pathway analysis showed a significant delay in osteoblast differentiation with GLI1 editing. Notably, while there was no significant difference in mesenchymal and endothelial cell survival during differentiation, the viability of MSCs differentiating toward osteocytes was significantly reduced.

We observed a significant down-regulation in genes of endodermal linage during spontaneous and directed differentiation of GLI1 edited cells compared to the WT control. Interestingly, the small molecule GLI inhibitor GANT-61 has been found to be successful in treating cancers originating in cells of endodermal lineage. Researchers have observed extensive cell death in a panel of 7 human colon carcinoma cell lines using GANT-61 ⁴⁸. GANT-61 was also found to be effective inhibiting the cancer initiating phenotype in lung adenocarcinoma cell lines, reviewed in ⁵³, although issues of bioavailability and toxicity have yet to be addressed.

Angiogenesis is critical to tissue growth and plays important roles in the pathogenesis of numerous diseases including cancer. Hypoxia is a key stimulus of angiogenesis and is mediated in part by HIF-1alpha and the oxytocin receptor. GANT-58 (a GLI1 inhibitor closely related to GANT-61) has been shown to abrogate oxytocin induced HIF-1alpha expression leading to reduction in angiogenic capacity of HUVEC cells⁵⁴. SHH signaling is known to promote vasculogenesis and angiogenesis⁵⁵ so down regulating the pathway by inhibiting GLI1 is expected to inhibit angiogenesis. GANT-61 also reduces proliferation, increases apoptosis and reduces stem cell markers in cancer cells⁵⁶. SHH/GLI inhibitors other than GANT also suppress tumor angiogenesis and tumor growth in xenograft models⁵⁷.

In mice, it was shown that even though elevated GLI1 expression leads to cell cycle arrest and apoptosis in neonatal NSCs, normal expression of GLI1 is necessary for their self-renewal⁵⁸. Studies on hESCs revealed that GLI1 was found to be a determinant of ventral floor plate specification and mesencephalic dopamine neuron generation during development 59. The human embryonic stem cell model showed that markers of neural differentiation PAX6 and SOX1 were expressed at lower levels with homozygous editing than in WT hESCs during the initiation of spontaneous and directed differentiation. Later stages of neural differentiation demonstrated reduction in expression of NES, VIM, and NEFM. Furthermore, edited cells had a reduced ability to form neurospheres. These results indicate that GLI1 editing delays early neural differentiation.

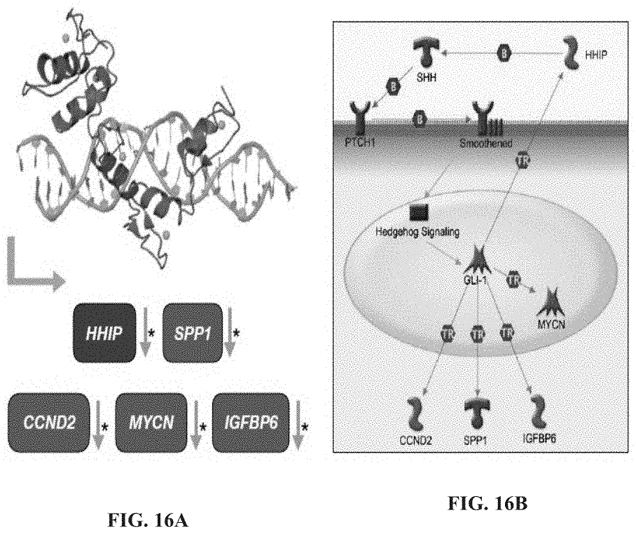

Our RNA-Seq data revealed alteration in expression of GLI1 target genes with roles in cell proliferation (HHIP, IGFBP6, MYCN, CCND2) and cell differentiation (SSP1). The HHIP gene is upstream of the Sonic Hedgehog (SHH) pathway. HHIP and downstream targets IGBFP6, MYCN, and CCND2 are transcriptionally up-regulated, which increases cellular proliferative potential. GLI1 is also a key player in cell fate determination during differentiation. The SPP1 gene, which is downstream of both SHH and BMP pathways, is transcriptionally activated by GLI1 to induce osteogenic differentiation. In the GLI1 edited cells, the expression levels of these target genes were significantly down-regulated (FIG. 16A-16B).

Transcriptional regulation occurs in a landscape of protein complexes that modulate the expression of genes. Key regulatory events occur in discrete areas that can coordinate the expression of dozens of genes and together with spatial control of enhancers orchestrate normal development. Alteration of the regulatory region is associated with disease burden. In the case of GLI1 the first intron represents such a region. Public data reveal dozens of transcription factors that bind this region. GLI transcription factors bind this region of the gene and along with BRD4 has been demonstrated, H2A.z and histone marks can account for much of the activity of the GLI1 positive feedback loop (Taylor, et al. 2019).

Summary

Our stem cell model based on editing of a regulatory region in the first intron of GLI1 highlight the importance of the six GBS in this region during stem cell differentiation and tissue development. RNA-Seq data and pathway analyses clearly show that GLI1 is highly involved in regulating stem cell differentiation toward all three embryonic lineages and plays a crucial role in vasculogenesis and hematopoiesis. These data suggest that manipulating this region can modulate GLI1 expression, which may provide new therapeutic strategies to treat human malignancies based on interrupting the positive GLI feedback loop.

### REFERENCES

1. Hui, C. C. & Angers, S. Gli proteins in development and disease. Annu Rev Cell Dev Biol 27, 513-537 (2011).
2. Robbins, D. J., Fei, D. L. & Riobo, N. A. The Hedgehog signal transduction network. Sci Signal 5, re6 (2012).
3. Villavicencio, E. H., Walterhouse, D. O. & Iannaccone, P. M. The sonic hedgehog-patched-gli pathway in human development and disease. Am J Hum Genet 67, 1047-1054 (2000).
4. Walterhouse, D. O., Lamm, M. L., Villavicencio, E. & Iannaccone, P. M. Emerging roles for hedgehog-patched-Gli signal transduction in reproduction. Biol Reprod 69, 8-14 (2003).
5. Palencia-Campos, A., et al. GLI1 inactivation is associated with developmental phenotypes overlapping with Ellis-van Creveld syndrome. Hum Mol Genet 26, 4556-4571 (2017).
6. Villavicencio, E. H., Walterhouse, D. O. & Iannaccone, P. M. The Sonic Hedgehog-Patched-Gli Pathway in Human Development and Disease. Am J Hum Genet 67(2000).
7. Walterhouse, D. O., Yoon, J. W. & Iannaccone, P. M. Developmental pathways: Sonic hedgehog-Patched-GLI. Environ Health Perspect 107, 167-171 (1999).
8. Iannaccone, P. M., et al. The Hedgehog Signaling Pathway in Development and Disease. in Inborn Errors of Development: The Molecular Basis of Clinical Disorders of Morphogenesis (eds. Epstein, C.J.n.e. & et al.) (2008).
9. Yu, M., et al. Sonic hedgehog-responsive genes in the fetal prostate. J Biol Chem 284, 5620-5629 (2009).
10. Yoon, J. W., Gilbertson, R., Iannaccone, S., Iannaccone, P. & Walterhouse, D. Defining a role for Sonic hedgehog pathway activation in desmoplastic medulloblastoma by identifying GLI1 target genes. Int J Cancer 124, 109-119 (2009).
11. Lamm, M. L., et al. Sonic hedgehog activates mesenchymal Gli1 expression during prostate ductal bud formation. Dev Biol 249, 349-366 (2002).
12. High, A. & Zedan, W. Basal cell nevus syndrome. Current opinion in oncology 17, 160-166 (2005).
13. Regl, G., et al. Human GLI2 and GLI1 are part of a positive feedback mechanism in Basal Cell Carcinoma. Oncogene 21, 5529-5539 (2002).
14. Yoon, J. W., et al. Gene expression profiling leads to identification of GLI1-binding elements in target genes and a role for multiple downstream pathways in GLI1-induced cell transformation. J Biol Chem 277, 5548-5555 (2002).
15. Carpenter, R. L., et al. The gain-of-function GLI1 transcription factor TGLI1 enhances expression of VEGF-C and TEM7 to promote glioblastoma angiogenesis. Oncotarget 6, 22653-22665 (2015).
16. Ali, S. A., Niu, B., Cheah, K. S. E. & Alman, B. Unique and overlapping GLI1 and GLI2 transcriptional targets in neoplastic chondrocytes. PLoS One 14, e0211333 (2019).
17. Xu, X., et al. Sonic Hedgehog-GLI Family Zinc Finger 1 Signaling Pathway Promotes the Growth and Migration of Pancreatic Cancer Cells by Regulating the Transcription of Eukaryotic Translation Initiation Factor 5A2. Pancreas 44, 1252-1258 (2015).
18. Ghanbari, A., Cheraghzadeh, Z., Mahmoudi, R., Zibara, K. & Hosseini, E. GLI inhibitors overcome Erlotinib resistance in human pancreatic cancer cells by modulating E-cadherin. J Chemother 31, 141-149 (2019).
19. Yoon, J. W., Lamm, M., Chandler, C., Iannaccone, P. & Walterhouse, D. Up-regulation of GLI1 in vincristine-resistant rhabdomyosarcoma and Ewing sarcoma. BMC Cancer 20, 511 (2020).
20. Gu, D. & Xie, J. Non-Canonical Hh Signaling in Cancer-Current Understanding and Future Directions. Cancers (Basel) 7, 1684-1698 (2015).
21. Yoon, J. W., et al. Noncanonical regulation of the Hedgehog mediator GLI1 by c-MYC in Burkitt lymphoma. Mol Cancer Res 11, 604-615 (2013).
22. Yuan, X., et al. Ciliary IFT80 balances canonical versus non-canonical hedgehog signalling for osteoblast differentiation. Nat Commun 7, 11024 (2016).
23. Zwerner, J. P., et al. The EWS/FLI1 oncogenic transcription factor deregulates GLI1. Oncogene 27, 3282-3291 (2008).
24. Taylor, R., et al. Regulation of GLI1 by cis DNA elements and epigenetic marks.

DNA repair 79, 10-21 (2019).
25. Chen, Q., et al. Fate decision of mesenchymal stem cells: adipocytes or osteoblasts?Cell Death Differ 23, 1128-1139 (2016).
26. Ahmed, S., et al. Transcription factors and neural stem cell self-renewal, growth and differentiation. Cell Adh Migr 3, 412-424 (2009).
27. Wu, S. M., Choo, A. B., Yap, M. G. & Chan, K. K. Role of Sonic hedgehog signaling and the expression of its components in human embryonic stem cells. Stem Cell Res 4, 38-49 (2010).
28. Po, A., et al. Hedgehog controls neural stem cells through p53-independent regulation of Nanog. EMBO J 29, 2646-2658 (2010).
29. Yoon, J. W., et al. p53 modulates the activity of the GLI1 oncogene through interactions with the shared coactivator TAF9. DNA repair 34, 9-17 (2015).
30. Bai, C. B., Auerbach, W., Lee, J. S., Stephen, D. & Joyner, A. L. Gli2, but not Gli1, is required for initial Shh signaling and ectopic activation of the Shh pathway. Development 129, 4753-4761 (2002).
31. Drakopoulou, E., et al. Non-redundant role for the transcription factor Gli1 at multiple stages of thymocyte development. Cell Cycle 9, 4144-4152 (2010).
32. Park, H. L., et al. Mouse Gli1 mutants are viable but have defects in SHH signaling in combination with a Gli2 mutation. Development 127, 1593-1605 (2000).
33. Thomson, J. A., et al. Embryonic stem cell lines derived from human blastocysts.

Science 282, 1145-1147 (1998).
34. Siller, R. & Sullivan, G. J. Rapid Screening of the Endodermal Differentiation Potential of Human Pluripotent Stem Cells. Current protocols in stem cell biology 43, 1g.7.1-1g.7.23 (2017).
35. Lian, X., et al. Efficient differentiation of human pluripotent stem cells to endothelial progenitors via small-molecule activation of WNT signaling. Stem cell reports 3, 804-816 (2014).
36. Choi, K. D., Vodyanik, M. & Slukvin, I I. Hematopoietic differentiation and production of mature myeloid cells from human pluripotent stem cells. Nature protocols 6, 296-313 (2011).
37. Yun, C., et al. Mechanistic insight into the effects of Aryl Hydrocarbon Receptor activation on osteogenic differentiation. Bone Rep 6, 51-59 (2017).
38. Galat, Y., et al. Cytokine-free directed differentiation of human pluripotent stem cells efficiently produces hemogenic endothelium with lymphoid potential. Stem Cell Research & Therapy 8(2017).
39. Li, Y., et al. Lysosomal transmembrane protein LAPTM4B promotes autophagy and tolerance to metabolic stress in cancer cells. Cancer Res 71, 7481-7489 (2011).
40. Beauchamp, E., et al. GLI1 is a direct transcriptional target of EWS-FLI1 oncoprotein. J Biol Chem 284, 9074-9082 (2009).
41. Campbell, V. T., et al. Hedgehog pathway inhibition in chondrosarcoma using the smoothened inhibitor IPI-926 directly inhibits sarcoma cell growth. Mol Cancer Ther 13, 1259-1269 (2014).
42. Houde, C., et al. Overexpression of the NOTCH ligand JAG2 in malignant plasma cells from multiple myeloma patients and cell lines. Blood 104, 3697-3704 (2004).
43. Merchant, A., Joseph, G., Wang, Q., Brennan, S. & Matsui, W. Gli1 regulates the proliferation and differentiation of HSCs and myeloid progenitors. Blood 115, 2391-2396 (2010).
44. Hojo, H., et al. Gli1 protein participates in Hedgehog-mediated specification of osteoblast lineage during endochondral ossification. J Biol Chem 287, 17860-17869 (2012).
45. Shi, Y., et al. Gli1 identifies osteogenic progenitors for bone formation and fracture repair. Nat Commun 8, 2043 (2017).
46. Galat, V., et al. Transgene Reactivation in Induced Pluripotent Stem Cell Derivatives and Reversion to Pluripotency of Induced Pluripotent Stem Cell-Derived Mesenchymal Stem Cells. Stem cells and development 25, 1060-1072 (2016).
47. Lauth, M., Bergstrom, A., Shimokawa, T. & Toftgard, R. Inhibition of GLI-mediated transcription and tumor cell growth by small-molecule antagonists. Proc Natl Acad Sci USA 104, 8455-8460 (2007).
48. Agyeman, A., Jha, B. K., Mazumdar, T. & Houghton, J. A. Mode and specificity of binding of the small molecule GANT61 to GLI determines inhibition of GLI-DNA binding.

Oncotarget 5, 4492-4503 (2014).
49. North, T., et al. Cbfa2 is required for the formation of intra-aortic hematopoietic clusters. Development 126, 2563-2575 (1999).
50. Sugimura, R., et al. Haematopoietic stem and progenitor cells from human pluripotent stem cells. Nature 545, 432-438 (2017).
51. Galat, Y., et al. Application of small molecule CHIR99021 leads to the loss of hemangioblast progenitor and increased hematopoiesis of human pluripotent stem cells. Experimental hematology 65, 38-48.e31 (2018).
52. Kitaura, Y., et al. Gli1 haploinsufficiency leads to decreased bone mass with an uncoupling of bone metabolism in adult mice. PLoS One 9, e109597 (2014).
53. Dimou, A., Bamias, A., Gogas, H. & Syrigos, K. Inhibition of the Hedgehog pathway in lung cancer. Lung Cancer 133, 56-61 (2019).
54. Zhu, J., Wang, H., Zhang, X. & Xie, Y. Regulation of angiogenic behaviors by oxytocin receptor through Gli1-induced transcription of HIF-1alpha in human umbilical vein endothelial cells. Biomed Pharmacother 90, 928-934 (2017).
55. Byrd, N. & Grabel, L. Hedgehog signaling in murine vasculogenesis and angiogenesis. Trends Cardiovasc Med 14, 308-313 (2004).
56. Po, A., et al. Noncanonical GLI1 signaling promotes stemness features and in vivo growth in lung adenocarcinoma. Oncogene 36, 4641-4652 (2017).
57. Lei, X., et al. Identification of a novel tumor angiogenesis inhibitor targeting Shh/Gli1 signaling pathway in Non-small cell lung cancer. Cell Death Dis 11, 232 (2020).
58. Galvin, K. E., Ye, H., Erstad, D. J., Feddersen, R. & Wetmore, C. Gli1 induces G2/M arrest and apoptosis in hippocampal but not tumor-derived neural stem cells. Stem Cells 26, 1027-1036 (2008).
59. Denham, M., et al. Gli1 is an inducing factor in generating floor plate progenitor cells from human embryonic stem cells. Stem Cells 28, 1805-1815 (2010).

All publications and patents mentioned herein are hereby incorporated by reference in their entirety as if each individual publication or patent was specifically and individually indicated to be incorporated by reference. In case of conflict, the present application, including any definitions herein, will control.

We claim:

1. A method of suppressing expression of GLI1 gene in a cell, said method comprising subjecting said cell using a CRISPR-Cas editing system and a guide RNA (gRNA) to generate suppress expression of GLI1; and treating a disease or disorder in a subject by administering to the subject a therapeutically effective amount of the GLI1 suppressed cell.

2. The method of claim 1, wherein the disease or the disorder is an infection, a cancer, a vascular disease.

3. The method of claim 2, wherein the cancer is a type of solid cancer, type of glioblastoma.

4. The method of claim 3, wherein the infection is a bacterial infection or a viral infection.

5. The method of claim 4, further comprising eliminating GLI1 positive feedback loop in hPSCs.

6. A method of treating a disease or disorder in a subject in need thereof comprising administering to a siRNA comprising the sequence selected from SEQ ID NO: 1-6 to generate a GLI modified cell; and treating a disease or disorder in a subject by administering to the subject a therapeutically effective amount of the GLI1 modified cell to treat the disease or disorder.

7. The method of claim 6, wherein the disease or the disorder is an infection, a cancer, a vascular disease.

8. The method of claim 7, wherein the cancer is a type of solid cancer, type of glioblastoma.

9. The method of claim 8, wherein the infection is a bacterial infection or a viral infection.

10. The method of claim 10, further comprising downregulating cancer-related gene expression to prevent tumor angiogenesis.
